(19) United States
(12) Patent Application Publication
(10) Pub. No.: US 2009/0186878 A1
(43) Pub. Date: Jul. 23, 2009

(54) CRYSTALLINE FORMS OF A FARNESYL DIBENZODIAZEPINONE

(75) Inventors: Patrick Morris, Lasalle (CA); Faustinus Yeboah, Longueuil (CA); James McAlpine, Montreal (CA); Maxime Ranger, Montreal (CA); Emmanuelle Roux, Montreal (CA); Michael Harvey, Kirkland (CA)

Correspondence Address: MERCHANT & GOULD PC, P.O. BOX 2903, MINNEAPOLIS, MN, 55402-0903, US

(73) Assignee: THALLION PHARMACEUTICALS INC., Montreal, QC (CA)

(21) Appl. No.: 12/162,631

(22) PCT Filed: Jan. 26, 2007
(86) PCT No.: PCT/US07/02291

§ 371 Date: Dec. 15, 2008

Related U.S. Application Data
(60) Provisional application No. 60/763,377, filed on Jan. 31, 2006.

(51) Int. Cl. A61K 31/55; C07D 243/38

(52) U.S. Cl. 514/220; 540/495

(57) ABSTRACT

The present invention relates to crystalline forms of ECO-4601 and the processes for providing them. The invention further relates to pharmaceutical compositions comprising the crystalline forms and to methods of use of the crystalline forms as pharmaceuticals.

### FIELD OF THE INVENTION

The present invention relates to crystalline forms of a farnesyl dibenzodiazepinone. The invention also relates to the process of preparing the crystalline forms, pharmaceutical compositions comprising the crystalline forms, and to the method of using them in a medicament for administration to a mammal in need of such medicament.

### BACKGROUND OF THE INVENTION

The novel farnesyl dibenzodiazepinone (herein referred to as Compound 1 as below) was isolated from novel strains of actinomycetes, Micromonospora sp. as disclosed in U.S. application Ser. No. 10/762,107 filed Jan. 21, 2004, also published as WO 2004/065591 in August 2004, incorporated herein by reference in their entirety. The structure was also disclosed in Charan et al. (2004), J. Nat. Prod., vol 67, 1431-1433 (as diazepinomicin), and in Igarashi et al. (2005), J. Antibiot., 350-352. This compound was found to have potent activities including anti-lipoxygenase, anti-bacterial and anti-cancer activities. Furthermore, U.S. application Ser. Nos. 10/951,436 (filed Sep. 27, 2004) and 11/130,295 (filed May 16, 2005) disclosed in vivo anti-cancer potency of the farnesyl dibenzodiazepinone in animal models. None of these disclosed either crystalline forms of Compound 1 or methods for producing them.

To prepare pharmaceutical compositions containing Compound 1 for administration to mammals, in accordance to health registration requirements of health registration authorities (e.g. FDA's Good Manufacturing Practices (GMP)), the compound should be used in a form as pure as possible, and having constant physical properties, including purity, solubility and stability. The solid-state properties of a drug alone or in the presence of excipients can have a very significant impact on the drug performances, including its stability, solubility, and bioavailability.

Compared to amorphous forms, crystalline forms generally have lower impurity concentration, and more consistent and uniform product quality, for example, more consistent physical characteristics such color, rate of dissolution and ease of handling, as well as longer-term stability. Thus, in the manufacture of a drug, a pharmaceutical composition or a medicament, it is important, whenever possible, to provide the active compound in a substantially crystalline form. Being more reliable, crystalline forms ensure a reproducibility of quality control results between batches, in terms of physical properties such as the melting point value.

A known case of polymorphism is the drug ritonavir, a protease inhibitor marketed for the treatment of HIV/AIDS by Abbott Laboratories under the trade name Norvir™. The product was first launched in 1996 under its only known solid form. A crystalline form was later discovered, which turned out to be thermodynamically more stable and 50% less soluble than the original form, and which was found to be forming during storage. This form did not meet regulatory dissolution specifications and the drug was withdrawn from the market. A soft gel formulation was re-launched with the second crystalline form but not without consequences to the company, and to HIV/AIDS patients for loss of treatment options.

Using a drug in an amorphous form having a glass transition (T_{g}) below 50° C. might be a concern for the development of solid oral dosage (see for example, Bechard and Down (1992), Pharmaceutical Research, vol 9, no 4, 521-528. Ideally, the form used should not have a T_{g} below 100° C. These are considered as standards in the industry, since amorphous forms having low T_{g}s are more prone to converting into a more thermodynamically stable form, which could happen for example, during production and formulation steps (e.g., heating, compressions, etc), during storage, or in the gastrointestinal track once administered.

### SUMMARY OF THE INVENTION

The present invention provides crystalline forms of Compound 1, methods for producing them and their use as pharmaceuticals. In one embodiment, Compound 1 has the following structural formula:

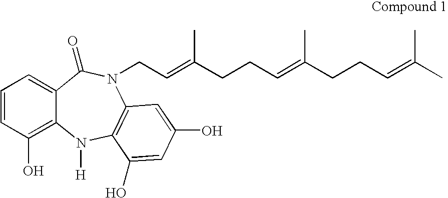

In one aspect, the invention provides a crystalline Form I. In one embodiment, crystalline Form I is characterized by a DSC (differential scanning calorimetry) scan showing at least a broad first-order transition phase between about 100° C. and about 140° C. and a melting temperature of about 183° C.±5° C. (onset by DSC), by an x-ray diffraction pattern essentially as shown in FIG. 1(c), and by a weight loss below 100° C. as shown by thermogravimetry analysis (TGA). In one embodiment, Form I is characterized by the following angular positions (two theta angles±1%) in a X-Ray powder diffraction pattern: 5.14°, 10.34°, 15.20°, 20.78°, 22.80°, 26.02° and 31.20°. In another embodiment, Form I is characterized by the following angular positions (two theta angles±1%) in a X-Ray powder diffraction pattern: 5.1°, 10.3°, 15.2°, 20.8°, 22.8°, 26.0° and 31.2°.

In another aspect, the invention provides a crystalline Form II. In one embodiment, crystalline Form II is characterized by a DSC scan showing a broad first-order transition phase between about 100 to about 140° C. and a melting temperature of about 183° C.±5° C. (onset by DSC), by an x-ray diffraction pattern essentially as shown in FIG. 1(b) or FIG. 1(d), and by a weight loss below 100° C. as shown by TGA. In one embodiment, Form II is characterized by the following angular positions (two theta angles±1%) in a X-Ray powder diffraction pattern: 4.16°, 8.32°, 12.50°, 16.70°, 20.94°, 25.20°, 29.48° and 33.82°. In another embodiment, Form II is characterized by the following angular positions (two theta angles±1%) in a X-Ray powder diffraction pattern: 4.2°, 8.3°, 12.5°, 16.7°, 20.9°, 25.2°, 29.5° and 33.8°.

In another aspect, the invention provides a crystalline Form III. In one embodiment, crystalline Form III is characterized by a DSC scan showing a melting temperature of about 183° C.±5° C. (onset temperature by DSC) and no first-order transition phase (no peak) before melting, and by an X-ray powder diffraction (XRPD) pattern essentially as shown in FIG. 1(a). In one embodiment, Form III is characterized by the following angular positions (two theta angles±1%) in a X-Ray powder diffraction pattern: 3.96°, 7.86°, 11.80°, 15.74°, 23.64° and 27.62°. In another embodiment, Form III is characterized by the following angular positions (two theta angles±1%) in a X-Ray powder diffraction pattern: 4.0°, 7.9°, 11.8°, 15.7°, 23.6° and 27.6°.

In another aspect, the invention provides a crystalline form of Compound 1 obtainable by crystallization from a solvent system comprising at least one lower alkyl alcohol. In one embodiment, the lower alkyl alcohol is a C₁₋₆ alkyl alcohol, preferably a C₁₋₄ alkyl alcohol. In another embodiment, the lower alkyl alcohol is selected from methanol, ethanol and isopropanol. In another embodiment, the solvent system comprises water and a lower alkyl alcohol selected from methanol, ethanol and isopropanol. In another embodiment, the crystalline form is Form I. In another embodiment, the crystalline form is Form II.

In another aspect, the crystalline form is obtainable by thermal treatment of a partly crystalline or substantially crystalline form. In one embodiment, the thermal treatment is done at a temperature ranging from about 50° C. to a temperature close to the melting point (e.g., about 170° C.), preferably about 50° C. to about 100° C., more preferably about 60° C. to about 80° C. In another embodiment, the thermal treatment is done for a period of 30 minutes to 24 hours, preferably 2 to 20 hours, more preferably 4 to 10 hours. In another embodiment, the thermal treatment is optionally accomplished under reduced pressure or under inert conditions.

In another aspect, the invention provides a method for preparing a crystalline form of Compound 1 comprising the steps of: (a) providing Compound 1, (b) treating Compound 1 with a solvent system, and (c) collecting the crystals. In one embodiment, step (b) of the method further comprises a decolorization step. In another embodiment, the method further comprises step (d): drying of the crystals collected in (c). In one embodiment, the solvent system comprises one or more solvent, which includes: organic solvents (e.g., lower alkyl alcohols, alkyl acetates, aliphatic hydrocarbons, halogenated hydrocarbons, lower dialkyl ketones, acetonitrile and dialkyl ethers) and aqueous solvents (e.g., water). Preferably, the solvent system includes a lower alkyl alcohol solvent, more preferably, the solvent system includes a lower alkyl alcohol and water. In a further embodiment, the thermal treatment of step (d) is done at a temperature ranging from about 50° C. to a temperature close to the melting point (e.g., about 170° C.), preferably about 50° C. to about 100° C., more preferably about 60° C. to about 80° C. In another embodiment, the thermal treatment is done for a period of about 30 minutes to about 24 hours, preferably about 2 to about 20 hours, more preferably about 4 to about 10 hours. In another embodiment, the thermal treatment of step (e) is optionally accomplished under reduced pressure or under inert conditions. In one embodiment, the crystals obtained in step (c) are of crystalline Form I. In another embodiment, the crystals obtained in step (c) are of crystalline Form II. In another embodiment, the crystals obtained in step (d) are of crystalline Form III.

In another aspect the invention provides pharmaceutical compositions comprising a therapeutically effective amount of at least one crystalline form of Compound 1, and a pharmaceutically acceptable carrier. In one embodiment, the pharmaceutical composition is for oral administration. In another embodiment, the pharmaceutical composition is a solid composition for oral administration. In another embodiment, the formulation is a liquid suspension. In a subclass of this embodiment, the liquid suspension is for intranasal, topical, oral, or parenteral administration, or for administration by inhalation. In yet another embodiment, the formulation is a solid formulation for oral administration or for administration by inhalation. In another embodiment, the pharmaceutical composition comprises crystalline Form I. In another embodiment, the pharmaceutical composition comprises crystalline Form II. In another embodiment, the pharmaceutical composition comprises crystalline Form III.

In another embodiment the invention provides the use of at least one crystalline form of Compound 1 in the preparation of a medicament for the treatment of a neoplasm in a subject in need of such treatment. In a subclass of this embodiment, the medicament is a solid oral composition or an oral suspension. In another embodiment the invention provides the use of at least one crystalline form of Compound 1 in the treatment of a neoplastic condition in a subject in need of such treatment. In another embodiment, the invention provides the use as antineoplastic agent, of a pharmaceutical composition comprising at least one crystalline form of Compound 1 and a pharmaceutically acceptable carrier. In another embodiment the invention provides the use of a pharmaceutical composition comprising at least one crystalline form of Compound 1 and a pharmaceutically acceptable carrier, in the preparation of a medicament to treat a neoplastic condition in a subject in need of such treatment. The invention further provides a kit or commercial package comprising at least one crystalline form of Compound 1 together with a written matter describing instructions for the use of Compound 1 crystals for treating a neoplastic condition.

In a further embodiment, the invention provides a method for the treatment of neoplasm comprising the step of administering a therapeutically effective amount of at least one crystalline form of Compound 1 to a subject in need of such treatment. In a further embodiment, the crystalline form of Compound 1 is administered as a pharmaceutical composition further comprising a pharmaceutically acceptable carrier.

In an embodiment, the neoplasm treated in any of the above mentioned method or use is selected from lung cancer, colorectal cancer (including colon cancer), CNS cancer (including glioma), ovarian cancer, renal cancer, prostate cancer, breast cancer, hematopoietic cancer (including leukemia) and melanoma.

### BRIEF DESCRIPTION OF FIGURES

FIG. 1(a-d): shows characteristic X-ray powder diffraction (XRPD) patterns (at room temperature) of the various Compound 1 crystalline forms [Vertical Axis: Intensity; horizontal Axis: Two theta (degrees) from 2 to 40 degrees], for peak values, see Table 1. FIG. 1(a) shows a characteristic XRPD pattern of crystalline Form III after annealing process at 60° C. for 6 hours. FIG. 1(b) shows a characteristic XRPD pattern of crystalline Form II (crystallized from isopropanol/water). FIG. 1(c) shows a characteristic X-ray powder diffraction pattern (at room temperature) of crystalline Form I (crystallized from methanol/water). FIG. 1(d) shows a characteristic XRPD pattern of crystalline Form II (crystallized from ethanol/water).

FIG. 2: shows a DSC (differential scanning calorimetry) thermogram of a partly amorphous powder form of Compound 1, with temperature ramp of 20° C./min, from −60° C. to 21° C., including a glass transition at −15° C.

FIGS. 3 to 5: All DSC thermograms shown in FIGS. 3 to 5 were accomplished from room temperature (25° C.) to 210° C. under nitrogen with a temperature ramp of 5° C./min.

FIG. 3(a,b): FIG. 3(a) shows a DSC thermogram of crystalline Form I, showing a broad first-order transition below a melting point of about 183° C. FIG. 3(b) shows a DSC thermogram of crystalline Form III after annealing of Form I at 60° C. for 6 hours under reduced pressure, showing no first-order transitions below a melting point of about 183° C.

FIG. 4(a,b): FIG. 4(a) shows a DSC thermogram of crystalline Form II (from ethanol/water), showing a first-order transition below a melting point of about 183° C. FIG. 4(b) shows a DSC thermogram of crystalline Form III after annealing of Form II (from ethanol/water) at 60° C. for 6 hours under reduced pressure, showing no first-order transitions below a melting point of about 183° C.

FIG. 5(a to d): shows a DSC thermogram of crystalline Form III after annealing of Form II (from ethanol/water) at different temperatures for 6 hours. FIG. 5(a) shows a DSC of Form III from annealing of Form II at 110° C. FIG. 5(b) shows a DSC of Form III from annealing of Form II at 90° C. FIG. 5(c) shows a DSC of Form III from annealing of Form II at 70° C. FIG. 5(d) shows a DSC of Form III from annealing of Form II at 60° C.

FIGS. 6 and 7: show TGA (thermogravimetry analysis) thermograms, with a temperature ramp of 20° C./min, from room temperature (25° C.) to 675° C. From room temperature (25° C.) to 550° C. with a nitrogen gas flow. At 550° C., nitrogen flow was changed to air flow for facilitating the final transition (degradation).

FIG. 6: FIG. 6(a) shows a TGA thermogram of Form III after annealing of Form I (from methanol/water) at 60° C. for 6 hours under reduced pressure. FIG. 6(b) shows a TGA thermogram of Form I (from methanol/water) showing a weight loss below 100° C.

FIG. 7: FIG. 7(a) shows a TGA thermogram of Form III after annealing of Form II (from ethanol/water) at 60° C. for 6 hours under reduced pressure. FIG. 7(b) shows a TGA thermogram of Form II (from ethanol/water) showing a weight loss below 100° C.

### DETAILED DESCRIPTION OF THE INVENTION

One aspect of the invention provides crystalline forms of the compound having a structural formula defined as Compound 1, which exhibits more reproducible purity and/or physical stability than the powder form, found to exhibit a glass transition (T_{g}) around −15° C. Crystalline forms of Compounds I (especially Forms I, II and III) and the Compound 1 powder as described in WO 2004/065591 and U.S. Ser. Nos. 10/951,436 (filed Sep. 27, 2004) and 11/130,295 (filed May 16, 2005) have comparable spectra of antitumor activity.

A second aspect of the invention provides processes for preparing the crystal forms of Compound 1. In one embodiment, the process provides for the step of collecting the crystallization product. In another embodiment, the process comprises crystallization of the compound performed in large scale for commercial production of Compound 1.

A third aspect of the invention provides methods for crystallizing Compound I. In one embodiment, the methods increase the purity and/or physical stability of the compound compared to the powder form of the compound before crystallization. The methods comprise the step of crystallizing the powder under conditions in which the crystallized compound is more pure than the amorphous preparation of the compound.

In another aspect of this embodiment, Compound 1 crystallizes to produce Form I. In another aspect of this embodiment, Compound 1 crystallizes to produce Form II. In a further aspect of this embodiment, thermal treatment of an essentially, substantially or partly crystalline form, for example containing Form I or II crystals or both, produces another crystalline form, for example Form III.

### I. DEFINITIONS

Unless otherwise defined all technical and scientific terms used herein have the meaning as commonly understood by a person skilled in art to which this invention belongs.

The term “farnesyl dibenzodiazepinone”, “compound”, “drug” or “active ingredient” shall mean Compound 1. The term “Compound 1”, when used in the context of a process or method for producing Compound 1 crystals, refers to Compound 1 as starting material for crystallization, which may be in a crude, powder, substantially pure or essentially pure form, it may be amorphous, partly crystalline or crystalline, as one crystal form or a mixture of forms may be used to produce the same (e.g. to further purify) or a different crystal form.

The purity of Compound 1 or its crystalline forms refers to the compound prior to its formulation in a pharmaceutical composition. The purity is referred to by “percent purity” and is a measure of the amount of Compound 1 (crystalline or not) relative to the presence of components other then Compound 1 and is not the measure of the degree of crystallization. The purity may be measured by means including nuclear magnetic resonance (NMR) spectroscopy, gas chromatography/mass spectroscopy (GC/MS), liquid chromatography/mass spectrometry (LC/MS), and liquid chromatography/UV spectroscopy (LC/UV).

The term “isolated” refers to a compound or product which has been removed from its original environment (e.g. reaction mixture, production culture or fermentation), which may be in a solid or powder form, a semi-solid form or an oily form and refers to a compound or product that is at least 5%, 10%, 20%, 30%, 40%, 50%, 60%, 70%, 80%, 90%, 95% (% by weight) of the compound present in the mixture. The term “crude” refers to a mixture of Compound 1 that contains at least 50% of Compound 1, by weight, it may be a semi-solid or oily form, or a solid or powder form, and may include crystals. The term “pure” or “purified” refers to substantially pure or essentially pure Compound 1. The term “substantially pure” refers to a sample having at least 95% wt of Compound 1. The term “essentially pure” refers to a sample having at least 97% wt of Compound 1.

The term “powder form” of Compound 1 refers to an amorphous or partly amorphous form, which may be partly crystalline. A powder form of Compound 1 will generally exhibit a glass transitions (T_{g}) at about −15° C. under the conditions described herein.

The term “crystal forms” or “polymorphs” generally refer to solid forms having the same chemical composition (e.g., Compound 1) but having different 3-dimensional arrangement, having or not solvent and/or water molecules included in said arrangement. The term “amorphous” generally refers to a form having little or no 3-dimensional arrangement.

The determination of Compound 1 as a crystal may be determined by means including optical microscopy, electron microscopy, x-ray powder diffraction, solid state NMR spectroscopy or polarizing microscopy. Optical and electron microscopy can also be used to determine the sizes and shapes of the crystals. The invention herein includes all crystals of Compound 1.

The term “crystalline Compound 1”, “Compound 1 crystals” or “crystal form(s) of Compound 1” refer to a solid form of Compound 1 comprising greater than 50%, 60%, 70%, 80%, 90% or 95% of one or more crystal forms or polymorphs of Compound 1. The term “substantially crystalline” refers to a solid form of Compound 1 comprising at least 95% of crystals of Compound 1. The term “essentially crystalline” refers to a crystalline form essentially free of amorphous forms.

The term “treating Compound 1” refers to crystallizing or recrystallizing Compound 1 from any of the solvents described herein. The necessary steps for crystallization or recrystallization are described in Section III.

The term “solute” refers to a substance that is dissolved in another substance to form a solution. As used herein, the solute refers to Compound 1, in an amorphous powder or crystalline form.

The term “solution” refers to two or more substances mixed to form a single, homogenous phase. One of the substances is the solvent and the others (solutes) are said to be dissolved in it. As used herein, the solution comprises one solute as described above, and one or more solvents in combination.

The term “low molecular weight alcohol”, “lower alkyl alcohol” or “C₁₋₆ alkyl alcohol” refers to an organic compound containing at least one alcohol functional group and 1 to 6 carbon atoms. Representative examples of low molecular weight alcohols include, without limitation, methanol, ethanol, propanols (e.g., iso and n-propanol), butanols (e.g., iso, sec, tert and n-butanol), and glycols (e.g. ethylene glycol and propylene glycol).

The term “first order transition temperature” refers to a temperature at which the physical state changes to another state (with no molecular degradation). This temperature can be a molecular rearrangement (change of crystal type) or a melting point. The term “melting point” refers to a temperature at which a solid matter (crystalline or amorphous) turns to liquid state.

The terms “glass transition temperature” or “T_{g}” refer to a temperature at which a change of calorific capacity occurs, and the solid matter (amorphous state) gains a degree of freedom in terms of molecular mobility.

The term “temperature ramp” refers to the heating and the cooling rate at which the scan is performed.

The term “weight loss” refers to the loss in weight of the sample (or its contents such as solvent) during a heating process using a constant temperature ramp and is usually expressed in % wt (% weight). This weight loss can be associated with the elimination of solvent(s), trapped in the compound. Weight loss is also associated with the molecular degradation of compound after solvent elimination.

The term “decomposition temperature” refers to the temperature at which a compound starts to degrade. It is a transition usually occurring after the melting point.

The term “annealing” refers a technique involving heating and controlled cooling of a material to increase the size of its crystals and reduce their defects. The heat causes the atoms (molecules) to become unstuck from their initial positions (a local minimum of the internal energy) and wander randomly through states of higher energy; the slow cooling gives them more chances of finding configurations with lower internal energy than the initial one. The terms “annealing process”, “annealing step” or “thermal treatment” refer to an isothermal step, during which the temperature is set constant for a determined period, in order to get a more stable crystalline state, sometimes accompanied by desolvation. The term “desolvation” refers to a process by which a composition is freed of the majority of solvent molecules respectively. When the solvent molecules are water molecules, the desolvation process is also called “dehydration”. Herein, “annealing” occurs during a simple drying process at an isothermal temperature, and the terms “annealing process”, “thermal treatment” and “drying” are equally used throughout the specification.

The term “Compound 1-producing microorganism” and equivalents refer to a microorganism that carries genetic information necessary to produce Compound 1, whether or not the organism naturally produces the compound. The terms apply equally to organisms in which the genetic information to produce Compound 1 is found in the organism as it exists in its natural environment, and to organisms (host cells) in which the genetic information is introduced by known recombinant techniques. Example of genetic information that can be introduced in a host cell is provided in US Patent publication no 2005-0043297 and U.S. patent application Ser. No. ______, filed Jan. 12, 2006, incorporated herein by reference in their entirety.

### II. CRYSTALLINE FORMS OF COMPOUND 1

The invention provides crystal forms of a compound herein referred as Compound 1:

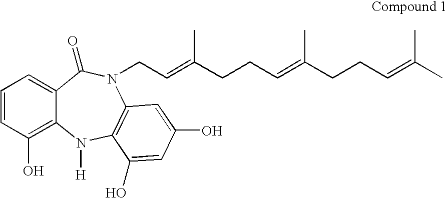

Crystalline Forms I, II and III of Compound 1 are characterized by any one or more of their physicochemical properties, such as: melting temperature, X-Ray powder diffraction (XRPD) pattern, Differential Scanning Calorimetry (DSC) thermogram, and Thermogravimetry analysis (TGA). All crystalline forms are characterized by a melting temperature of about 183° C.±5° C. (onset by DSC).

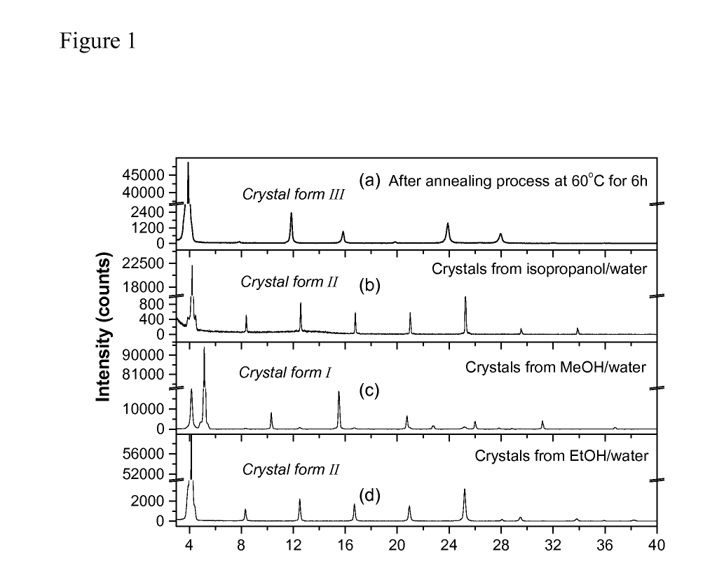

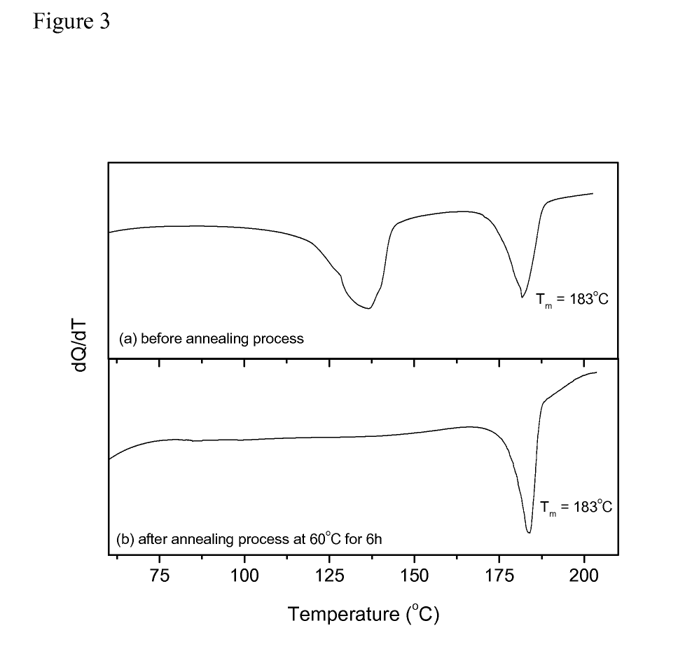

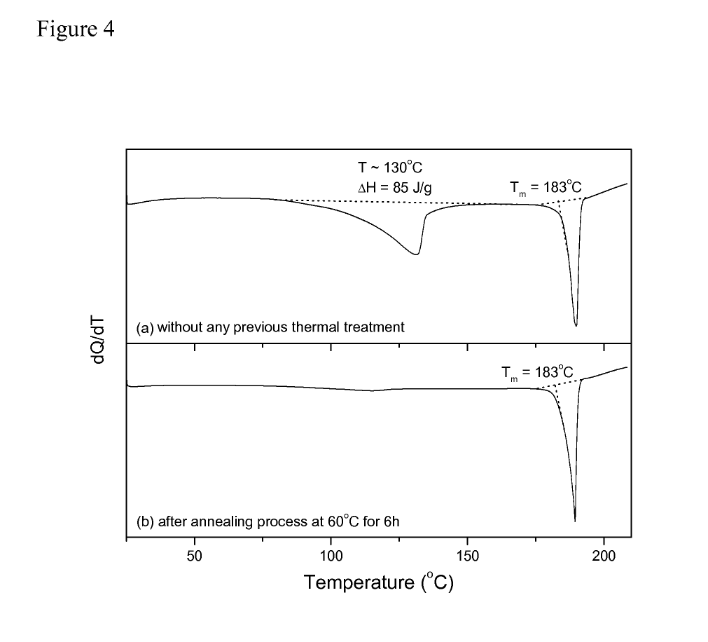

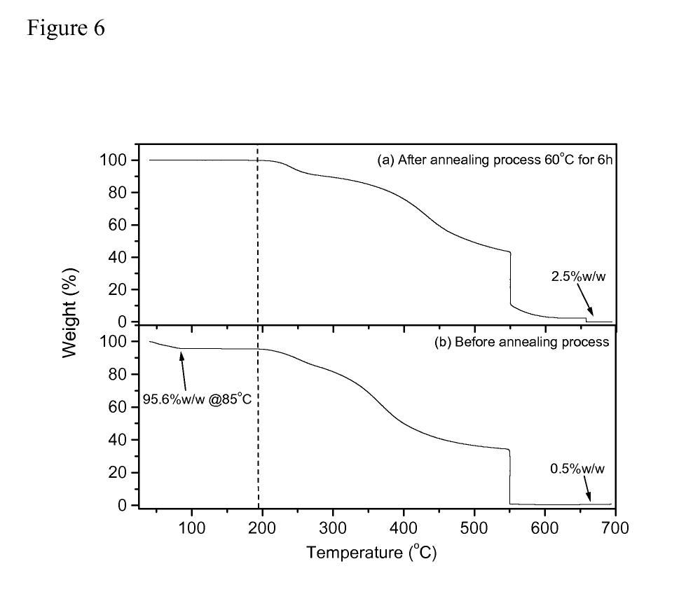

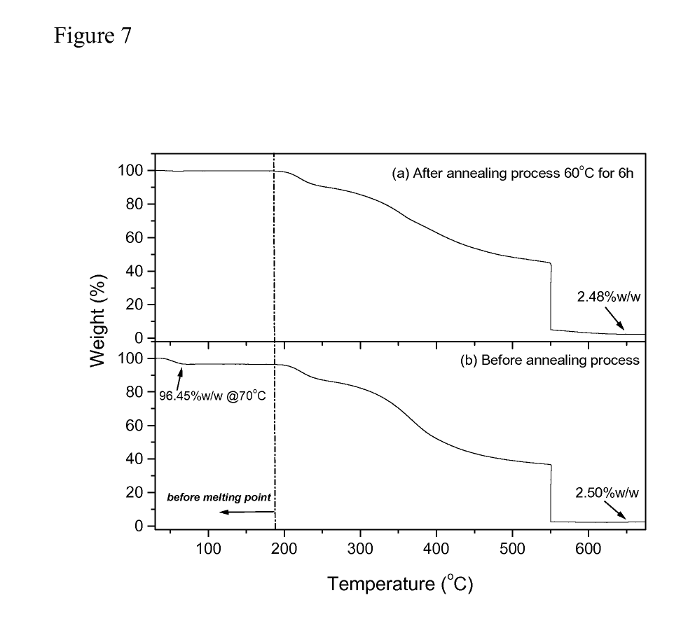

Crystal Form I is further characterized by: an XRPD pattern essentially as shown in FIG. 1(c) and as described in Table 1, a DSC thermogram essentially as shown in FIG. 3(a), at least a broad first-order transition between about 100° C. and about 140° C., a melting temperature of about 183° C.±5° C. (onset by DSC), and a TGA thermogram essentially as shown in FIG. 6(b). Crystal Form II is further characterized by: an XRPD pattern essentially as shown in FIG. 1(b) or FIG. 1(d) and as described in Table 1, a DSC thermogram essentially as shown in FIG. 4(a), a broad first-order transition between about 100 to about 140° C., a melting temperature of about 183° C.±5° C. (onset by DSC), a TGA thermogram essentially as shown in FIG. 7(b).

Crystal Form III is further characterized by: an XRPD pattern essentially as shown in FIG. 1(a) and as described in Table 1, a DSC thermogram essentially as shown in any of FIGS. 3(b), 4(b) and 5(a-d), no first-order transition below melting point, a melting temperature of about 183° C.±5° C. (onset by DSC), and a TGA thermogram essentially as shown in any of FIGS. 6(a) and 7(a).

Crystalline Forms I and II are produced as described herein by treatment of Compound 1 with a lower alkyl alcohol, and Form III is produced by drying Form I or II. All forms may be produced by treatment of Compound 1 with different solvent systems. The crystalline forms of the invention are not limited to the process by which they are produced as exemplified herein. Form III may be produced by treatment of Compound 1 with an aprotic solvent system. Form III may be produced by treatment of Compound 1 with an aprotic solvent system and by evaporating the solvents under reduced pressure, with gentle warming.

### III. METHODS FOR PRODUCING THE CRYSTALS

Crystalline forms of drugs are generally obtained by methods such as melting and slow cooling, by crystallization (sometimes called recrystallization), by sublimation or by thermal treatment, sometimes involving a dehydration or desolvation step. Crystallization is generally accomplished by different methods, depending on the properties of the starting material (e.g., degree of crystallinity, purity, impurities present, solubility, stability, etc), these methods include, for example, classical crystallization using a single solvent (poor solvent at low temperature but good solvent when heated), by achieving the same result by using co-solvents (e.g., at least one good and one poor solvent), by cooling a solution, by seeding crystals of the compound or by slow evaporation.

Crystallization is generally made possible by the phenomenon of supersaturation. Supersaturation is a condition under which the amount of solute dissolved in a solvent is more than the solvent can hold. For example, a solid substance containing a major component “solute A” and a minor impurity “solute B” (A and B having similar solubility properties) is dissolved in a hot solvent, such that the solution is saturated in solute A but not in solute B. As the solution is allowed to cool, it reaches a point where the solution becomes supersaturated in solute A, and crystals of solute A begin to form slowly at first. The initial crystal acts as seed that induces further crystallization of solute A from solution, while solute B, which is not at a point of saturation remains in solution. The pure crystals of solute A can than be recovered.

Supersaturation is attained by different methods. Generally, when a saturated solution is cooled, or when solvent from the solution slowly evaporates, the solution becomes supersaturated. Other methods, such as the addition of an anti-solvent (poor solvent), or by decreasing solubility by the addition of a salt, such as NaCl or triethylamine hydrochloride, or by combinations of any of the above mentioned methods.

The use of a pure solvent for crystallization is limited, since the solvent has to exhibit a large solubility difference over a narrow temperature range. The use of a solvent system is more flexible. This solvent system comprises at least one good solvent (a solvent in which the drug has good solubility) and at least one poor solvent (a solvent in which the drug is poorly soluble), which is miscible in the first one. The solvent system usually comprises one good solvent and one poor solvent (anti-solvent), but may also include more than two solvents.

Examples of good solvents for dissolving Compound 1 include, without limitation, lower alkyl alcohols (e.g., methanol, ethanol, isopropanol, n-butanol, propylene glycol), acetonitrile, aromatic solvents (e.g., toluene) and oxygen containing organic solvents such as dialkyl ketones (e.g., acetone, 2-butanone), tetrahydrofuran, dioxane, alkyl acetates (e.g., ethyl acetate, iso-propyl acetate, butyl acetate), and dialkyl ethers (e.g., tert-butyl methyl ether). Examples of solvents in which Compound 1 is poorly soluble include, without limitation, aqueous solvents (e.g. water), aliphatic hydrocarbons (e.g. hexanes, n-heptane, iso-octane) and halogenated hydrocarbons (e.g., dichloromethane, chloroform).

At any step of the process, prior to crystal formation, the solution can be treated with a decolorizing agent such as Norit™ charcoal, followed by filtration of said agent. The decolorization step is also done by passing the solution through a column of decolorizing agent, with or without the aid of a filtering agent, such as Celite™. The decolorizing step is accomplished as the drug is in solution, prior to the crystallization process, for example, before the addition of the poor solvent of a co-solvents system, or if the solution is heated, before the cooling process.

The crystallization is initiated when supersaturation is attained. The crystals may form naturally or the process may be initiated by the use of precipitating agents, or by seeding the crystals of the compound in the solution. Slow evaporation of solvent or addition of a small quantity of poor solvent may also initiate crystallization. A change in pH or addition of salts can also aid the crystallization process. Crystals are collected by standard techniques including filtration, centrifugation and decantation or combinations thereof.

The crystals are produced as solvate forms (including hydrate forms when the solvent is water), or as substantially ansolvate forms (with no or very few solvent molecules present in the crystal structure, also called anhydrate when the absent solvent is water). Solvate forms are prepared in the presence solvent (or water molecules for hydrate forms). Anhydrate forms are prepared by excluding water from the solvent system (e.g., by using substantially water-free organic solvents), or by warming the hydrated crystals until entrapped water molecules are eliminated. Accordingly, ansolvate forms are prepared by using solvents which would not stay within the crystal structure, by using solvent systems substantially excluding solvents having the tendency to stay entrapped, or by warming (drying) solvated crystals until entrapped solvent molecules are eliminated.

Crystals are obtained in different forms or polymorphs, following the conditions used for their preparation (e.g. temperature, solvents and solvent proportions, and concentration of the compound). The forms differ by the three-dimensional arrangement of molecules in the crystals, and usually by the presence or absence of water and/or solvent molecules in the crystal structure. These differences are shown by analyzing the crystals by x-ray powder diffraction, or by optical microscopy, electron microscopy, solid state NMR spectroscopy or polarizing microscopy. The different polymorphs are of various energy and stability. Polymorphic forms can sometimes transform in another crystalline form (e.g., a lower energy or more stable form) by methods such as drying and thermal treatments, also referred to as annealing processes.

Crystalline forms can have any macroscopic crystalline or crystal-like shape including without limitation, needle-like, rod-like, plate-like, flake-like or urchin-like such that urchin like means needle-like crystals grouped together to resemble a sea urchin.

General Procedure for the Production of Compound 1 Crystals:

In one aspect, the invention provides methods for producing Compound 1 in a substantially crystalline form, the method comprising the steps of (a) providing Compound 1, (b) treating Compound 1 with a solvent system comprising one or more solvent, and (c) separating the crystals from the supernatant. In one embodiment, step (b) comprises a decolorization step. In another embodiment, step (b) comprises seeding Compound 1 crystals. In another embodiment, the method further optionally comprises step (d), annealing or drying the crystals separated in (c).

Compound 1 prior to crystallization is provided in a crude, powder, amorphous or partly amorphous form and may also be partly crystalline, or crystalline. Compound 1 may be a powder or crude form of Compound 1, or a substantially pure form. A crystalline form may also be produced by treating Compound 1, wherein Compound 1 is the same or a different crystalline form, or a mixture of crystalline forms. A powder or crude form of Compound 1 may be obtained by cultivation of a “Compound 1-producing microorganism”, followed by isolation and purification techniques including precipitation, filtration, HPLC (high performance liquid chromatography), High Speed Counter Current chromatography, size exclusion ultrafiltration and/or ion exchange chromatography, and techniques using other resins, including Diaion™ HP-20 column. Exemplary procedures to produce Compound 1 are provided in Example 1.

In one aspect, Compound 1 is treated with a solvent system comprising at least one good solvent and one poor solvent. In another aspect, the solvent system comprises a lower alkyl alcohol, preferably the lower alkyl alcohol is selected from methanol, ethanol or isopropanol, using water as “antisolvent” (poor solvent). Times and temperatures of crystallization depend on the concentration and purity of Compound 1 in solution, and on the solvent system used. Purity of the crystals obtained may depend on the purity of the starting material, i.e. the purity of Compound 1 prior to crystallization. Crystals are preferably separated by filtration, but are also collected by other means such as centrifugation and/or decantation.

In another aspect of the invention, the process for producing crystalline forms includes step (d) drying at least one crystalline form of Compound 1 at an isothermal temperature of about 50° C. to about 170° C., preferably between about 50 and about 100° C., more preferably between about 60 and about 80° C., for about 30 minutes to about 24 hours, preferably between about 2 to about 20 hours, and most preferably around about 4 to about 10 hours. In one embodiment, the drying step is optionally done under inert conditions such as reduced pressure or inert atmosphere (e.g., nitrogen or argon atmosphere). In one embodiment, the crystalline form before drying is Form I or Form II, or a mixture thereof. In another embodiment, the crystalline form obtained after drying is Form III.

In a further aspect, the method may be accomplished on large scale, process or production scale, using any pharmaceutically accepted equipment or method, known to the art of pharmaceutical production.

In another aspect, the invention provides a method for preparing a crystalline form of Compound 1 comprising the steps of: (a) providing Compound 1, (b) treating Compound 1 with a solvent system comprising water and at least one lower alkyl alcohol, and (c) collecting the crystals formed. In one embodiment, step (b) further comprises a decolorization step. In another embodiment, the method further comprises step (d) drying the crystals collected in (c). In one embodiment, the ratio of lower alkyl alcohol to water is between 20:80 to 80:20, preferably of about 30:70 to 60:40. In another embodiment, the final concentration of powder in the alcohol/water solvent system is about 0.1 to 100 mg/mL, preferably about 15 to 100 mg/L.

In another aspect, the invention provides a method for preparing a crystalline form of Compound 1 comprising the steps of: (a) providing Compound 1, (b) treating Compound 1 with a solvent system comprising about 20% to about 80% of ethanol in water, (c) warming the solution at a temperature of about 27° C. to 40° C. until enough ethanol has evaporated to attain supersaturation, and (d) collecting the crystals formed. In one embodiment, the method further comprises step (e) drying the crystals collected in (d).

Because of their high purity and easy handling, the crystalline forms of this invention may be used for the preparation of a medicament. They may be used as intermediates in a process for the preparation of a medicament for parenteral or non-parenteral administration.

### IV. PHARMACEUTICAL COMPOSITIONS COMPRISING A CRYSTALLINE FORM

The invention provides a pharmaceutical composition comprising at least one crystalline form of Compound 1, as described herein, in combination with a pharmaceutically acceptable carrier. The pharmaceutical composition comprising a crystal form is useful for treating diseases and disorders associated with uncontrolled cellular growth and proliferation, such as a neoplastic condition. The pharmaceutical composition comprising a crystal form of Compound 1 may be packaged into a convenient commercial package providing the necessary materials, such as the pharmaceutical composition and written instructions for its use in treating a neoplastic condition, in a suitable container.

The crystals of the invention may be further processed before formulation. For example, crystalline forms of Compound 1 may be milled or ground into smaller particles before appropriate formulation.

The crystals of the present invention can be formulated for oral, sublingual, intranasal, intraocular, rectal, transdermal, mucosal, topical or parenteral administration for the therapeutic or prophylactic treatment of neoplastic and proliferative diseases and disorders. Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracerebral or intracranial, intraspinal, intracisternal, and intrathecal (spinal fluids). Any known device useful for parenteral injection or infusion of drug formulations can be used to effect such administration. For oral and/or parental administration, crystal forms of the present invention can be mixed with conventional pharmaceutical carriers and excipients and used in the form of solutions, emulsions, tablets, capsules, soft gels, elixirs, suspensions, syrups, wafers and the like. The formulation can be a solid formulation used, for example, in oral, sublingual, or rectal administration. The compositions comprising a crystal form of the present invention will contain from about 0.1% to about 99.9%, about 1% to about 98%, about 5% to about 95%, about 10% to about 80% or about 15% to about 60% by weight of the crystal form.

The pharmaceutical compositions disclosed herein are prepared in accordance with standard procedures (USP, FDA) and are administered at dosages that are selected to reduce, prevent, or eliminate cancer or pre-cancer. (See, e.g., Remington's Pharmaceutical Sciences, Mack Publishing Company, Easton, Pa.; and Goodman and Gilman, Pharmaceutical Basis of Therapeutics, Pergamon Press, New York, N.Y., the contents of which are incorporated herein by reference, for a general description of the methods for administering various medicaments for human therapy, including chemotherapy).

As used herein, the term “unit dosage” refers to physically discrete units suitable as unitary dosages for human subjects and other mammals, each unit containing a predetermined quantity of a crystal form (active ingredient) calculated to produce the desired therapeutic effect, in association with a suitable pharmaceutically acceptable carrier. In one embodiment, the unit dosage contains from 10 to 3000 mg of active ingredient. In another embodiment, the unit dosage contains 20 to 1000 mg of active ingredient. The compositions of the present invention can be delivered using controlled (e.g., capsules) or sustained release delivery systems (e.g., bioerodable matrices). Exemplary delayed release delivery systems for drug delivery that are suitable for administration of the compositions of the invention are described in U.S. Pat. Nos. 4,452,775 (issued to Kent), 5,039,660 (issued to Leonard), and 3,854,480 (issued to Zaffaroni), incorporated herein by reference in their entirety.

The pharmaceutically-acceptable compositions of the present invention comprise one or more crystal forms of the present invention in association with one or more non-toxic, pharmaceutically-acceptable carriers and/or diluents and/or adjuvants and/or excipients, collectively referred to herein as “carrier” materials, and if desired other active ingredients. Pharmaceutically acceptable carriers include, for example, solvents, vehicles or medium such as saline, buffered saline, dextrose, water, glycerol, ethanol, propylene glycol, polysorbate 80 (Tween-80™), poly(ethylene) glycol 300 and 400 (PEG 300 and 400), PEGylated castor oil (E.g. Cremophor EL), poloxamer 407 and 188, hydrophobic carriers, and combinations thereof. Hydrophobic carriers include, for example, fat emulsions, lipids, PEGylated phopholids, polymer matrices, biocompatible polymers, lipospheres, vesicles, particles, and liposomes. The term specifically excludes cell culture medium.

Excipients or additives included in a formulation have different purposes depending, for example on the nature of the drug, and the mode of administration. Examples of generally used excipients include, without limitation: stabilizing agents, solubilizing agents and surfactants, buffers, antioxidants and preservatives, tonicity agents, bulking agents, lubricating agents, emulsifiers, suspending or viscosity agents, inert diluents, fillers, disintegrating agents, binding agents, wetting agents, lubricating agents, antibacterials, chelating agents, sweetners, perfuming agents, flavouring agents, coloring agents, administration aids, and combinations thereof.

The compositions may contain common carriers and excipients, such as cornstarch or gelatin, lactose, sucrose, microcrystalline cellulose, kaolin, mannitol, dicalcium phosphate, sodium chloride and alginic acid. The compositions may contain crosarmellose sodium, microcrystalline cellulose, sodium starch glycolate and alginic acid.

Formulations for parenteral administration can be in the form of aqueous or non-aqueous isotonic sterile injection solutions, suspensions or fat emulsions, comprising at least one crystal form of Compound 1. The parenteral form used for injection must be fluid to the extent that easy syringability exists. These solutions or suspensions can be prepared from sterile concentrated liquids, powders or granules. The crystals can be dissolved in a carrier such as a solvent or vehicle, for example, polyethylene glycol, propylene glycol, ethanol, corn oil, benzyl alcohol, glycofurol, N,N-dimethylacetamide, N-methylpyrrolidone, glycerine, saline, dextrose, water, glycerol, hydrophobic carriers, and combinations thereof.

The formulation of the crystalline form may be prepared as a suspension for parenteral or non-parenteral administration, for example oral, intranasal or topical. When particle size reduction of the crystalline form is necessary, it may be achieved by mechanical means like milling or grinding, or by micronisation. Crystalline particles are also produced using apparatus and methods known in the art, for example using continuous flow cells, such as described in International Patent Application WO/38811. Additional excipients may also be used in the suspension preparation, such as suspending agents, surface stabilizers, dispersing agents, etc. Examples of suspending agents include, but are not limited to, carboxymethylcellulose, veegum, tragacanth, bentonite, methylcellulose, microcrystalline cellulose and polyethylene glycols. Depending on the mode of administration, the maximum particles average size needed may vary, for example from about 50 nm to about 100 μm. When used for non-parenteral administration, the particles average size may be higher than for parenteral administration.

Excipients used in parenteral preparations also include, without limitation, stabilizing agents (e.g. carbohydrates, amino acids and polysorbates), solubilizing agents (e.g. cetrimide, sodium docusate, glyceryl monooleate, polyvinylpyrolidone (PVP) and polyethylene glycol (PEG)) and surfactants (e.g. polysorbates, tocopherol PEG succinate, poloxamer and Cremophor™), buffers (e.g. acetates, citrates, phosphates, tartrates, lactates, succinates, amino acids and the like), antioxidants and preservatives (e.g. BHA, BHT, gentisic acids, vitamin E, ascorbic acid and sulfur containing agents such as sulfites, bisulfites, metabisulfites, thioglycerols, thioglycolates and the like), tonicity agents (for adjusting physiological compatibility), suspending or viscosity agents, antibacterials (e.g. thimersol, benzethonium chloride, benzalkonium chloride, phenol, cresol and chlorobutanol), chelating agents, and administration aids (e.g. local anesthetics, anti-inflammatory agents, anti-clotting agents, vaso-constrictors for prolongation and agents that increase tissue permeability), and combinations thereof.

Parenteral formulations using hydrophobic carriers include, for example, fat emulsions and formulations containing lipids, lipospheres, vesicles, particles and liposomes. Fat emulsions include in addition to the above-mentioned excipients, a lipid and an aqueous phase, and additives such as emulsifiers (e.g. phospholipids, poloxamers, polysorbates, and polyoxyethylene castor oil), and osmotic agents (e.g. sodium chloride, glycerol, sorbitol, xylitol and glucose). Liposomes include natural or derived phospholipids and optionally stabilizing agents such as cholesterol The parenteral unit dosage form of the compound can be a ready-to-use solution of the crystals in a suitable carrier in sterile, hermetically sealed ampoules or in sterile pre-loaded syringes. The suitable carrier optionally comprises any of the above-mentioned excipients.

Alternatively, the unit dosage of the crystals of the present invention can be in a concentrated liquid, powder or granular form for ex tempore reconstitution in the appropriate pharmaceutically acceptable carrier at the time of delivery. In addition the above-mentioned excipients, powder forms optionally include bulking agents (e.g. mannitol, glycine, lactose, sucrose, trehalose, dextran, hydroxyethyl starch, ficoll and gelatin), and cryo or lyoprotectants.

For example, in intravenous (IV) use, a sterile formulation of a crystal form of Compound 1 and optionally one or more additives, including solubilizers or surfactants, can be dissolved or suspended in any of the commonly used intravenous fluids and administered by infusion. Intravenous fluids include, without limitation, physiological saline, phosphate buffered saline, 5% glucose or Ringer's™ solution.

In another example, in intramuscular preparations, a sterile formulation of the crystals of the present invention can be dissolved and administered in a pharmaceutical diluent such as Water-for-Injection (WFI), physiological saline or 5% glucose. A suitable insoluble form of the crystals of Compound 1 may be prepared and administered as a suspension in an aqueous base or a pharmaceutically acceptable oil base, e.g. an ester of a long chain fatty acid such as ethyl oleate.

For oral use, solid formulations such as tablets and capsules are particularly useful. Sustained released or enterically coated preparations may also be devised. For pediatric and geriatric applications, suspension, syrups and chewable tablets are especially suitable. For oral administration, the pharmaceutical compositions are in the form of, for example, tablets, capsules, suspensions or liquid syrups or elixirs, wafers and the like. For general oral administration, excipient or additives include, but are not limited to inert diluents, fillers, disintegrating agents, binding agents, wetting agents, lubricating agents, sweetening agents, flavoring agents, coloring agents and preservatives.

The oral pharmaceutical composition is preferably made in the form of a unit dosage containing a therapeutically-effective amount of the active ingredient. Examples of such dosage units are tablets and capsules. For therapeutic purposes, the tablets and capsules which can contain, in addition to the active ingredient, conventional carriers such as: inert diluents (e.g., sodium and calcium carbonate, sodium and calcium phosphate, and lactose), binding agents (e.g., acacia gum, starch, gelatin, sucrose, polyvinylpyrrolidone (Providone), sorbitol, or tragacanth methylcellulose, sodium carboxymethylcellulose, hydroxypropyl methylcellulose, and ethylcellulose), fillers (e.g., calcium phosphate, glycine, lactose, maize-starch, sorbitol, or sucrose), lubricants or lubricating agents (e.g., magnesium stearate or other metallic stearates, stearic acid, polyethylene glycol, waxes, oils, silica and colloical silica, silicon fluid or talc), disintegrants or disintegrating agents (e.g., potato starch, corn starch and alginic acid), flavouring, coloring agents, or acceptable wetting agents. Carriers may also include coating excipients such as glyceryl monostearate or glyceryl distearate, to delay absorption in the gastrointestinal tract.

Oral liquid preparations, generally in the form of aqueous or oily solutions, suspensions, emulsions, syrups or elixirs, may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

For both liquid and solid oral preparations, flavoring agents such as peppermint, oil of wintergreen, cherry, grape, fruit flavoring or the like can also be used. It may also be desirable to add a coloring agent to make the dosage form more aesthetic in appearance or to help identify the product. For topical use the crystals of present invention can also be prepared in suitable forms to be applied to the skin, or mucus membranes of the nose and throat, and can take the form of creams, ointments, liquid sprays or inhalants, lozenges, or throat paints. Such topical formulations further can include chemical compounds such as dimethylsulfoxide (DMSO) to facilitate surface penetration of the active ingredient. For application to the eyes or ears, the crystalline form of Compound 1 can be formulated in a liquid or semi-liquid form in hydrophobic or hydrophilic bases as ointments, creams, lotions, paints or powders. For rectal administration the crystals of the present invention can be administered in the form of suppositories admixed with conventional carriers such as cocoa butter, wax or other glycerides.

### V. METHOD OF INHIBITING TUMOR GROWTH

In one aspect, the invention relates to a method for inhibiting growth and/or proliferation of cancer cells in a mammal. In another aspect, the invention provides a method for treating neoplasms in a mammal. Mammals include ungulates (e.g. sheeps, goats, cows, horses, pigs), and non-ungulates, including rodents, felines, canines and primates (i.e. human and non-human primates). In a preferred embodiment, the mammal is a human.

As used herein, the terms “neoplasm”, “neoplastic disorder”, “neoplasia” “cancer,” “tumor” and “proliferative disorder” refer to cells having the capacity for autonomous growth, i.e., an abnormal state of condition characterized by rapidly proliferating cell growth which generally forms a distinct mass that show partial or total lack of structural organization and functional coordination with normal tissue. The terms are meant to encompass hematopoietic neoplasms (e.g. lymphomas or leukemias) as well as solid neoplasms (e.g. sarcomas or carcinomas), including all types of pre-cancerous and cancerous growths, or oncogenic processes, metastatic tissues or malignantly transformed cells, tissues, or organs, irrespective of histopathologic type or stage of invasiveness. Hematopoietic neoplasms are malignant tumors affecting hematopoietic structures (structures pertaining to the formation of blood cells) and components of the immune system, including leukemias (related to leukocytes (white blood cells) and their precursors in the blood and bone marrow) arising from myeloid, lymphoid or erythroid lineages, and lymphomas (relates to lymphocytes). Solid neoplasms include sarcomas, which are malignant neoplasms that originate from connective tissues such as muscle, cartilage, blood vessels, fibrous tissue, fat or bone. Solid neoplasms also include carcinomas, which are malignant neoplasms arising from epithelial structures (including external epithelia (e.g., skin and linings of the gastrointestinal tract, lungs, and cervix), and internal epithelia that line various glands (e.g., breast, pancreas, thyroid). Examples of neoplasms that are particularly susceptible to treatment by the methods of the invention include leukemia, and hepatocellular cancers, sarcoma, vascular endothelial cancers, breast cancers, central nervous system cancers (e.g. astrocytoma, gliosarcoma, neuroblastoma, oligodendroglioma and glioblastoma), prostate cancers, lung and bronchus cancers, larynx cancers, oesophagus cancers, colon cancers, colorectal cancers, gastrointestinal cancers, melanomas, ovarian and endometrial cancer, renal and bladder cancer, liver cancer, endocrine cancer (e.g. thyroid), and pancreatic cancer.

The compound is brought into contact with or introduced into a cancerous cell or tissue. In general, the methods of the invention for delivering the pharmaceutical compositions (comprising a crystalline form of the invention) in vivo utilize art-recognized protocols for delivering therapeutic agents with the only substantial procedural modification being the substitution of the crystal form of the present invention for the therapeutic agent in the art-recognized protocols. The route by which the crystal-containing formulation is administered, as well as the formulation, carrier or vehicle will depend on the location as well as the type of the neoplasm. A wide variety of administration routes can be employed. The formulation may be administered by intravenous or intraperitoneal infusion or injection. For example, for a solid tumor or neoplasm that is accessible, the formulation may be administered by injection directly into the tumor or neoplasm. For a hematopoietic neoplasm the formulation may be administered intravenously or intravascularly. For neoplasms that are not easily accessible within the body, such as metastases or brain tumors, the formulation may be administered in a manner such that it can be transported systemically through the body of the mammal and thereby reach the neoplasm and distant metastases for example orally, intrathecally, intravenously or intramuscularly. The crystal-containing formulation can also be administered orally, subcutaneously, intraperitoneally, topically (e.g., for melanoma), rectally (e.g., for colorectal neoplasm), vaginally (e.g., for cervical or vaginal neoplasm), nasally or by inhalation spray (e.g., for lung neoplasm).

The crystalline Compound 1-containing formulation is administered in an amount that is sufficient to inhibit the growth or proliferation of a neoplastic cell, or to treat a neoplastic disorder. The term “inhibition” refers to suppression, killing, stasis, or destruction of cancer cells. The inhibition of mammalian cancer cell growth according to this method can be monitored in several ways. Cancer cells grown in vitro can be treated with the crystalline form and monitored for growth or death relative to the same cells cultured in the absence of the crystalline form. A cessation of growth or a slowing of the growth rate (i.e., the doubling rate), e.g., by 50% or more at 100 micromolar, is indicative of cancer cell inhibition (see Anticancer Drug Development Guide: preclinical screening, clinical trials and approval; B. A. Teicher and P. A. Andrews, ed., 2004, Humana Press, Totowa, N.J.). Alternatively, cancer cell inhibition can be monitored by administering the pharmaceutical formulation to an animal model of the cancer of interest. Examples of experimental non-human animal cancer models are known in the art and described below and in the examples herein. A cessation of tumor growth (i.e., no further increase in size) or a reduction in tumor size (i.e., tumor volume by least a 58%) in animals treated with the formulation relative to tumors in control animals not treated with the formulation is indicative of significant tumor growth inhibition (see Anticancer Drug Development Guide: preclinical screening, clinical trials and approval; B. A. Teicher and P. A. Andrews, ed., 2004, Humana Press, Totowa, N.J.).

The term “treatment” refers to the application or administration of a crystalline Compound 1-containing formulation to a mammal, or application or administration of a formulation to an isolated tissue or cell line from a mammal, who has a neoplastic disorder, a symptom of a neoplastic disorder or a predisposition toward a neoplastic disorder, with the purpose to cure, heal, alleviate, relieve, alter, ameliorate, improve, or control the disorder, the symptoms of disorder, or the predisposition toward disorder. The term “treating” is defined as administering, to a mammal, an amount of a crystalline Compound 1-containing formulation sufficient to result in the prevention, reduction or elimination of neoplastic cells in a mammal (“therapeutically effective amount”). The therapeutically effective amount and timing of dosage will be determined on an individual basis and may be based, at least in part, on consideration of the age, body weight, sex, diet and general health of the recipient subject, on the nature and severity of the disease condition, and on previous treatments and other diseases present. Other factors also include the route and frequency of administration, the activity of the administered compound, the metabolic stability, length of action and excretion of the compound, drug combination, the tolerance of the recipient subject to the compound and the type of neoplasm or proliferative disorder. In one embodiment, a therapeutically effective amount of the compound is in the range of about 0.01 to about 750 mg/kg of body weight of the mammal. In another embodiment, the therapeutically effective amount is in the range of about 0.01 to about 300 mg/kg body weight per day. In yet another embodiment, the therapeutically effective amount is in the range of 10 to about 120 mg/kg body weight per day. The therapeutically effective doses of the above embodiments may also be expressed in milligrams per square meter (mg/m²) in the case of a human patient. Conversion factors for different mammalian species may be found in: Freireich et al, Quantitative comparison of toxicity of anticancer agents in mouse, rat, dog, monkey and man, Cancer Chemoth. Report, 1966, 50(4): 219-244). When special requirements may be needed (e.g. for children patients), the therapeutically effective doses described above may be outside the ranges stated herein. Such higher or lower doses are within the scope of the present invention.

To monitor the efficacy of tumor treatment in a human, tumor size and/or tumor morphology is measured before and after initiation of the treatment, and treatment is considered effective if either the tumor size ceases further growth, or if the tumor is reduced in size, e.g., by at least 10% or more (e.g., 20%, 30%, 40%, 50%, 60%, 70%, 80%, 90% or even 100%, that is, the absence of the tumor). Prolongation of survival, time-to-disease progression, partial response and objective response rate are surrogate measures of clinical activity of the investigational agent. Tumor shrinkage is considered to be one treatment-specific response. This system is limited by the requirement that patients have visceral masses that are amenable to accurate measurement. Methods of determining the size of a tumor in vivo vary with the type of tumor, and include, for example, various imaging techniques well known to those in the medical imaging or oncology fields (MRI, CAT, PET, etc.), as well as histological techniques and flow cytometry. For certain types of cancer, evaluation of serum tumor markers are also used to evaluate response (eg prostate-specific antigen (PSA) for prostate cancer, and carcino-embryonic antigen (CEA), for colon cancer). Other methods of monitoring cancer growth include cell counts (e.g. in leukemias) in blood or relief in bone pain (e.g. prostate cancer).

The crystalline Compound 1-containing formulation may be administered once daily, or may be administered as two, three, four, or more sub-doses at appropriate intervals throughout the day. In that case, the amount of Compound 1 contained in each sub-dose must be correspondingly smaller in order to achieve the total daily dosage. The dosage unit can also be compounded for delivery over several days, e.g., using a conventional sustained release formulation which provides sustained release of the compound over a several day period. Sustained release formulations are well known in the art. In this embodiment, the dosage unit contains a corresponding multiple of the daily dose. The effective dose can be administered either as a single administration event (e.g., a bolus injection) or as a slow injection or infusion, e.g. over 30 minutes to about 24 hours. The formulation may be administered as a treatment, for up to 30 days. Moreover, treatment of a subject with a therapeutically effective amount of a composition can include a single treatment or a series of treatments (e.g., a four-week treatment repeated 3 times, with a 2 months interval between each treatment). Estimates of effective dosages, toxicities and in vivo half-lives for the compounds encompassed by the invention are made using conventional methodologies or on the basis of in vivo testing using an appropriate animal model.

Treatment of tumor in a subject, including mammals and humans, may be accomplished by administering the formulation of the invention as a single agent, or in combination with surgery and/or known anticancer treatments such as radiotherapy and chemotherapy regimen. The crystalline Compound 1 may be administered in conjunction with or in addition to known anticancer compounds or chemotherapeutic agents. Chemotherapeutic families include: cytostatic or cytotoxic agents, antibiotic-type agents, alkylating agents, antimetabolite agents, hormonal agents, aromatase agents, immunological agents, interferon-type agents, cyclooxygenase inhibitiors (e.g. COX-2 inhibitors), matrix metalloprotease inhibitors, telomerase inhibitors, tyrosine kinase inhibitors, anti-growth factor receptor agents, anti-HER agents, anti-EGFR agents, anti-angiogenesis agents, farnesyl transferase inhibitors, ras-raf signal transduction pathway inhibitors, cell cycle inhibitors, other CDK inhibitors, tubulin binding agents, topoisomerase I inhibitors, topoisomerase II inhibitors, and the like. Examples of chemotherapeutic agents include, but are not limited to, 5-fluorouracil, mitomycin C, methotrexate, hydroxyurea, nitrosoureas (e.g., BCNU, CCNU), cyclophosphamide, procarbazine, dacarbazine, thiotepa, atreptozocine, temozolomide, enzastaurin, erlotinib, mitoxantrone, anthracyclins (Epirubicin and Doxurubicin), CPT-11, camptothecin and derivatives thereof, etoposide, navelbine, vinblastine, vincristine, pregnasone, platinum compounds such as carboplatin and cisplatin, taxanes such as taxol and taxotere; hormone therapies such as tamoxifen and anti-estrogens; antibodies to receptors, such as herceptin and Iressa; aromatase inhibitors, progestational agents and LHRH analogs; biological response modifiers such as IL2 and interferons; multidrug reversing agents such as the cyclosporin analog PSC 833. (For more examples, see: The Merck Index, 12^{th} edition (1996), Therapeutic Category and Biological Activity Index, lists under “Antineoplastic” sections.

Toxicity and therapeutic efficacy of Compound 1 crystals can be determined by standard pharmaceutical procedures in cell cultures or experimental animals. Therapeutic efficacy is determined in animal models as described above and in the examples herein. Toxicity studies are done to determine the lethal dose for 10% of tested animals (LD10). Animals are treated at the maximum tolerated dose (MTD): the highest dose not producing mortality or greater than 20% body weight loss. The effective dose (ED) is related to the MTD in a given tumor model to determine the therapeutic index of the compound. A therapeutic index (MTD/ED) close to 1.0 has been found to be acceptable for some chemotherapeutic drugs, a preferred therapeutic index for classical chemotherapeutic drugs is 1.25 or higher.

The data obtained from cell culture assays and animal studies can be used in formulating a range of dosage for use in humans. The dosage of compositions of the invention will generally be within a range of circulating concentrations that include the MTD. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound. Such information can be used to more accurately determine useful doses in humans. Levels in plasma may be measured, for example, by high performance liquid chromatography. Animal models to determine antitumor efficacy of a compound are generally carried out in mice. Either murine tumor cells are inoculated subcutaneously into the hind flank of mice from the same species (syngeneic models) or human tumor cells are inoculated subcutaneously into the hind flank of severe combined immune deficient (SCID) mice or other immune deficient mouse (nude mice) (xenograft models).

Advances in mouse genetics have generated a number of mouse models for the study of various human diseases including cancer. The MMHCC (Mouse models of Human Cancer Consortium) web page (emice.nci.nih.gov), sponsored by the National Cancer Institute, provides disease-site-specific compendium of known cancer models, and has links to the searchable Cancer Models Database (cancermodels.nci.nih.gov), as well as the NCI-MMHCC mouse repository. Mouse repositories can also be found at: The Jackson Laboratory, Charles River Laboratories, Taconic, Harlan, Mutant Mouse Regional Resource Centers (MMRRC) National Network and at the European Mouse Mutant Archive. Such models may be used for in vivo testing of Compound 1 crystals, as well as for determining a therapeutically effective dose.

### EXAMPLES

Unless otherwise indicated, reagents and solvents used in the following examples were supplied by Sigma-Aldrich and or Fisher Scientific. Unless otherwise indicated, all numbers expressing quantities of ingredients, properties such as crystallization conditions, molecular weight, melting points, X-Ray powder diffractogram data such as relative intensity and distances values and so forth used in the specification and claims are to be understood as being modified in all instances by the term “about”. Accordingly, unless indicated to the contrary, the numerical parameters set forth in the present specification and attached claims are approximations. At the very least, and not as an attempt to limit the application of the doctrine of equivalents to the scope of the claims, each numerical parameter should at least be construed in light of the number of significant figures and by applying ordinary rounding techniques. Notwithstanding that the numerical ranges and parameters setting forth the broad scope of the invention are approximations, the numerical values set in the examples, Tables and Figures are reported as precisely as possible. Any numerical values may inherently contain certain errors resulting from variations in experiments, testing measurements, statistical analyses and such.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

### Example 1

### Production and Isolation of Compound 1

Compound 1 to be used in the crystallization of the invention is produced and isolated from “Compound 1-producing microorganisms”. The procedures provided in Example 1 are only provided as exemplary procedures and are not intended to be limiting.

Compound 1 was obtained according to the procedures described in Examples 1 and 2 of U.S. application Ser. No. 10/762,107 filed Jan. 21, 2004, also published as WO 2004/065591 in August 2004, using Micromonospora strains [S01]046 or 046-ECO11 having respectively IDAC accession numbers 231203-01 and 070303-01 (International Depository Authority of Canada (IDAC), Bureau of Microbiology, Health Canada, 1015 Arlington Street, Winnipeg, Manitoba, Canada, R3E 3R2).

Additionally, Compound 1 was produced as follows:

1.1. Procedure 1

A. Fermentation:

The fermentation was accomplished as a 1×10 L batch in a 14.5 L fermentor (BioFlo 110™ Fermentor, New Brunswick Scientific, Edison, N.J., USA) using an improved procedure described in U.S. patent application Ser. No. 10/762,107.

Micromonospora sp. (deposit accession number IDAC 070303-01) was maintained on agar plates of ISP2 agar (Difco Laboratories, Detroit, Mich.). An inoculum for the production phase was prepared by transferring the surface growth of the Micromonospora sp. from the agar plates to 2-L flasks containing 500 mL of sterile KH medium. Each liter of KH medium comprises 10 g glucose, 20 g potato dextrin, 5 g yeast extract, 5 g NZ-Amine A, and 1 g CaCO₃ made up to one liter with water (pH 7.0). The culture was incubated at about 28° C. for approximately 70 hours on a rotary shaker set at 250 rpm. Following incubation, 300 mL of culture was transferred to a 14.5 L fermentor containing 10 L of sterile production medium HI. Each liter of production medium HI was composed of 20 g potato dextrin, 30 g glycerol, 2.5 g Bacto-peptone, 8.34 g yeast extract, and 3 g CaCO₃, (with 0.3 mL Silicone defoamer oil (Chem Service) and 0.05 ml Proflo Oil™ (Traders protein) as antifoam agents, only when used in fermentor), made to one liter with distilled water and adjusted to pH 7.0. The culture was incubated at 28° C., with dissolved oxygen (dO₂) controlled at 25% in a cascade loop with agitation varied between 150-450 RPM and aeration set at a fixed rate of 0.5 V/V/M.

B. Isolation of Compound 1:

At harvest, the pH of the culture broth (1×10 L) was adjusted to 3.0 by the drop-wise addition of 20% aqueous H₂SO₄ (sulfuric acid) and with constant stirring. The resulting mixture was cooled to 4° C. and held at that temperature for 12 h. The cooled broth was then centrifuged (3200 rpm for 20 min) to separate mycelia. The mycelia recovered was extracted with methanol (2×300 mL of MeOH for every 100 g of mycelia).

After extraction, methanol extracts were pooled and evaporated to dryness under reduced pressure using a rotary evaporator. The methanolic extract concentrate was reconstituted in MeOH (100 mL for every 10 g of concentrate) and the resulting solution was transferred into a separating funnel. Distilled water (30 mL for every 10 g of concentrate) followed by hexanes (50 mL for every 10 g of concentrate) was then added to the methanolic solution in the separating funnel. The mixture was gently agitated by swirling, to allow for good phase contact but avoid emulsion formation. The mixture was then allowed to stand for phase separation to occur. The upper hexane layer was discarded. The aqueous methanol layer was recovered into a separating funnel, an equal volume of 15% NaCl and twice the volume of EtOAc (ethyl acetate) were added. The resulting mixture was swirled to allow good phase contact and allowed to stand for phase separation to occur. The upper EtOAc layer was recovered. Diaion™ HP-20 resin was added to the EtOAc layer and the solvent was removed under reduced pressure to allow binding of solute to the resin.

The solute-bound resin was applied to a Diaion™ HP-20 column and eluted with water to remove water soluble components, followed by 60% aqueous MeOH (v/v) to remove weakly bound impurities. The target compound was then eluted with a stepwise gradient of 80% to 90% aqueous MeOH. The 80-90% aqueous MeOH fractions were pooled and concentrated to dryness in vacuo to give crude Compound 1.100 mg of the crude Compound 1 was digested in 5 mL of the upper phase of a mixture prepared from chloroform, cyclohexane, methanol, and water in the ratios, by volume, of 5:2:10:5. The sample was subjected to centrifugal partition chromatography using a High Speed Countercurrent (HSCC) system (Kromaton Technologies, Angers, France) fitted with a 200 mL cartridge and prepacked with the upper phase of this two-phase system. The HSCC was run with the lower phase as mobile and Compound 1 was eluted at approximately one-half column volume. Fractions were collected and Compound 1 was detected by TLC of aliquots of the fractions on commercial Kieselgel 60F₂₅₄ plates. Compound could be visualized by inspection of dried plates under UV light or by spraying the plates with a spray containing vanillin (0.75%) and concentrated sulfuric acid (1.5%, v/v) in ethanol and subsequently heating the plate. Fractions containing Compound 1, were pooled and concentrated to yield a substantially pure, although highly colored, Compound 1.

1.2. Procedure 2

A. Fermentation

Micromonospora sp. [S01U02]046 (IDAC 070905-01) was maintained on GYM agar plates. The surface growth was transferred to three 2 L baffled flasks containing 500 mL of sterile KH medium each (see Example 1.1A) and grown for 70 to 72 hours at 28±1.0° C. on an orbital shaker. Seed flasks were pooled and transferred aseptically to a 28 L capacity inoculum fermentor. The volume transferred corresponded to 3% of the volume of KH medium (see Ex. 1.1A, in fermentor, KH further comprises as antifoam agents: 0.3 mL Silicone defoamer oil (Chem Service) and 0.05 ml Proflo Oil™ (Traders protein)) in the inoculum fermentor. Fermentation was performed at 28±1.0° C. for 48 hours, with dissolved oxygen maintained at 25% linked to agitation.

The entire volume from the inoculum fermentor was transferred to a 750 L capacity pilot fermentor. The volume transferred corresponded to 3.3% of the volume of medium HI (see Ex. 1.1A, including the antifoam agents) in the pilot fermentor. Fermentation was performed at 28±1.0° C. for 96 hours, with dissolved oxygen maintained at 25% linked to agitation.

B. Isolation of Compound 1

Prior to harvesting the pilot fermentor, the pH of the broth was adjusted to pH 3 by a slow addition of 99% H₂SO₄ with constant stirring. The fermentation culture was then cooled to 4±2° C. in the fermentation vessel and subsequently transferred into a holding tank and held at 4±2° C. for 16 to 72 hours. The mycelia was then harvested by ultrafiltration (0.2 micron filter membrane) to produce a thick slurry.

For every 1 L of mycelial slurry obtained after the mycelia separation step, 3 L of methanol was utilized. The extraction step involved the circulation of the mycelia-methanol mixture 60±10 min through the ultrafiltration system. The high circulating speed allowed breaking up of mycelial aggregates and the temperature was allowed to increase to 42±3° C., providing for an efficient extraction. Once the mycelia was properly mixed with the extraction solvent, the valves of the ultrafiltration system were opened to allow the permeate to be collected (clear methanolic extract). The methanol extract was fed into a retentate container and the residual mycelia were re-extracted with a second equivalent volume of methanol. This step was repeated with a third equivalent volume of methanol. The three methanolic extracts were pooled and evaporated under reduced pressure to produce a thick crude concentrate.

At room temperature, salt (NaCl 10% w/v) was added to the crude concentrate and the mixture was stirred for 30±10 min to allow dissolution of the salt. Methanol was added to the salinated crude concentrate at a ratio of 3:1 (methanol:concentrate) and the mixture was stirred for 60±10 min. The resulting mixture was filtered (0.5 micron membrane) under vacuum to remove particulate matter. The filtrate was transferred into a separating vessel followed by the addition of heptane (50 mL of heptane per 100 mL of methanol used to re-dissolve the crude concentrate). The content of the separating container was stirred well for 20±5 min to ensure complete contact of the aqueous and organic phases. After stirring the mixture is allowed to stand for phase separation to occur at room temperature. The lower aqueous methanol layer was collected. The methanolic extract was re-extracted with a second volume of heptane (an amount equal to 50% of the volume of heptane used in the first extraction). The methanolic layers were pooled.

The methanol layer obtained from defatting was mixed with HP20® in a rotary evaporator, and the methanol was evaporated under reduced pressure to allow hydrophobic components to bind to the resin. The loaded resin was added onto a pre-packed HP20® column. The column was washed extensively with purified water (10±2 column volumes) to remove any solvents, salts, and unbound water-soluble organic components. Weakly bound impurities which were less hydrophobic than Compound 1 were eluted from the column with aqueous methanol (60:40 v/v methanol:water), approximately 10±2 column volumes until the column effluent color was clear or very light yellow. A 70:30 methanol:water solution (3±1 column volumes) was then used to wash the column. Compound 1 was eluted with aqueous:methanol (90:10 v/v methanol:water), and fractions were collected. A sample of each 90% elution fraction was submitted for LC-UV analysis to determine Compound 1 content. The 70% and 90% aqueous methanol fraction containing greater than 1% of the total estimated amount of Compound 1 were pooled and submitted to a second HP20® column clean up, proceeding as described before. The resultant 90:10 v/v methanol:water fractions containing greater than 1% of the estimated amount of Compound 1 were pooled and concentrated to dryness prior to crystallization.

### Example 2

### Preparation of Compound 1 Crystals

The crystallization process is not limited to the use of isolated, crude or powder forms of Compound 1, crystalline forms can also be used in the crystallization process, to produce either the same or a different form. The same crystal forms may also be obtained from other solvent systems or under different conditions, the procedures exemplified herein are only for the purpose of illustrating.

Compound 1 lyophilized powder obtained according to Example 1 was used in the preparation of crystals of Form I and crystals of Form II (except for 2.1C were crude material was used). Crystals of Form III were prepared from the crystals of Form I or Form II.

2.1. Crystal Form I (Methanol/Water)(3 Procedures):

Note: traces of Form II crystals were sometimes present in Form I crystals produced from a methanol and water mixture. Production of Form I crystals was also observed when treating Compound 1 at about 0.8 mg/mL with a mixture of PEG (polyethylene glycol) and PG (propylene glycol), each at a concentration of 3% w/v in water.

A. Lyophilized Compound 1 (24 mg) from HSCC purification in Example 1.1B was weighed in a 20 mL glass vial and dissolved in 2 mL of methanol to produce a light brownish solution. The solution was passed over a plug of Norit™ (activated charcoal) in a Pasteur pipette to decolorize the solution. A light yellowish solution was obtained and a few drops of methanol were added to adjust the volume to 2 mL. The decolorized solution was titrated with water until the solution just turned cloudy. Constant swirling was used during titration. The total volume of water added was about 700 μL for a final methanolic content of about 72%. The cloudy suspension was heated to 55° C. in a water bath to produce a clear saturated solution. The clear solution was removed from the water bath and allowed to cool to room temperature. As the solution cooled, a supersaturated solution resulted, from which crystals started forming. The temperature of the solution at this point was about 31-33° C. The solution was allowed to stand in the dark at room temperature for 72 hours for complete crystallization to occur prior to filtration and washing in a sintered glass funnel. The crystals were lyophilized overnight to give 20.5 mg of crystalline Compound 1 (Form I).

B. An alternate procedure used was the following: Lyophilized Compound 1 (130 mg) from HSCC purification (Example 1.1B) was weighed and dissolved in 30 mL of methanol to produce a light brownish solution. The solution was passed over a short column of Norit™ (made from 200 mg of Norit and 400 mg of Celite as a filter aid) to decolorize the solution, vacuum pressure was used to facilitate the flow of the solution through the column. A light yellowish solution was obtained. An additional 5 mL of hot methanol was used to elute the column. The volume collected from the column was 34.2 mL. The decolorized solution was allowed to cool to room temperature and titrated with water until the solution began to turn cloudy. Constant swirling was used during titration. The total volume of water added was about 13 mL for a final methanolic content of 72%. The cloudy suspension was heated to 50° C. in a water bath to produce a clear saturated solution. The clear solution was removed from the water bath and allowed to cool gradually to room temperature in a beaker of water (water in the beaker had an initial temperature of 50° C.). Crystals began to appear in the solution after standing undisturbed for about 30 minutes (temperature in the beaker was 35° C.). The solution was allowed to stand in the dark at room temperature overnight, and then was put in the fridge (4° C.) for complete crystallization. The crystals were collected by filtration in a sintered glass funnel and washed with cold (4° C.) 20% aqueous methanol. The crystals were lyophilized overnight to give 116.3 mg of crystalline Compound 1 (Form I).

C. An alternate procedure was also used to prepare Form I crystals. The material used was obtained from the Diaion HP-20 step (Example 1.1B prior to HSCC). Approximately 1000 mg of lyophilized crude Compound 1 extract (about 70% purity) was dissolved in methanol (30 mL) to produce a dark brown colored solution (almost black). The solution was passed through a short column of Norit™ (made from 2 g Norit™ and 2 g Celite as filter aid) using vacuum to facilitate flow of solution. Initially, the solution eluted from the column as a light yellowish solution but the color changed to yellowish brown toward the end of elution. The Norit™ column was washed with 20 mL of hot methanol. The volume of the solution was adjusted to 50 mL with methanol to give a greenish yellow solution. The solution was allowed to cool to room temperature and titrated with water to cloud point, in a dropwise fashion and with constant swirling (20 mL of water was required, for a methanol concentration of about 71%). The cloudy suspension was heated to 50° C. in a water bath to produce a clear saturated solution. The resulting solution was removed from the water bath and allowed to cool to room temperature in a beaker of water (initial temperature of the beaker was 50° C.). Needle-like crystals began to appear in the solution after standing undisturbed overnight (crystals appeared after 15 hours). Drops of water were added to the solution to determine if the crystallization process was complete (cloudiness when water was added would mean crystallization was not complete). No significant cloudiness was observed so the solution was stored in the fridge (4° C.) for 5 hours. Crystals were collected by vacuum filtration using a sintered glass funnel, and washed with ice-cold 20% aqueous methanol. The recovered crystals (Form I) were lyophilized and gave 350 mg of crystals (>98% purity by NMR and HPLC).

2.2. Crystal Form II (Ethanol/Water):

A. Lyophilized Compound 1 (110 mg) from HSCC purification (Example 1.1B) was weighed in a 20 mL vial and dissolved with 10 mL of ethanol to produce a brownish solution. The solution was titrated to the cloud point with water and constant swirling (14 mL of water were used, to give a 39% ethanol concentration). The cloudy suspension was heated to 50° C. in a water bath to produce a clear supersaturated solution. Plate-like specks of silvery crystals appeared in the solution upon standing unperturbed for about 2 hours. After completion of the crystallization process, the crystals were recovered and weighed as described earlier. A quantity of 95 mg of crystals (Form II) was recovered. The crystals had a silver-grey hue.

B. Alternatively, crystallization was performed on HP20®-purified material from Example 1.2B. HP20®-purified material was dissolved in 95% ethanol to a concentration of about 24±3 g/L. Purified water was added to obtain a 17.5±2.5 g/L “stock solution” in 70% ethanol. This solution was added to a 33% ethanol solution prepared in a carboy vessel preheated to about 30±2° C. Addition of the “stock solution” was achieved using a solvent delivery system set at 10 mg/min/l OL crystallization volume with constant stirring. After about 6±0.5 h the crystallization tank was seeded with 10 mg of Compound 1 crystals. Once the stock solution had been completely delivered into the crystallization solution, the system was allowed to mature for about 12.0±0.5 h. After the maturation period, purified water (0.15×total volume of crystallization solution) was added to the tank at a rate of 0.1×volume of crystallization solution. After the addition of purified water, the resulting crystallization solution was matured for an additional 16.0±0.5 h prior to the harvest of crystals. Crystals were collected by filtration using a medium gauge sintered glass funnel.

2.3 Crystal Form II (Isopropanol/Water):

Lyophilized Compound 1 (21 mg) from HSCC purification (Example 1.1B) was weighed in a borosilicate glass tube (13×100 mm) and dissolved with 800 μL of isopropyl alcohol. The solution was brought near the cloud point by adding water (1500 μL of water were used, to give a 35% isopropyl alcohol concentration). Solution was kept at 4-8° C. overnight to allow crystal formation. The crystals were recovered and weighed. The recovery yield was about 75%.

2.4 Crystal Form III (Annealing Process):

Crystal Form III was produced by annealing of either Form I or Form II crystals using a variety of temperatures and under various conditions such as air or inert atmosphere or under reduced pressure. The results are summarized below.

A. Example Procedure:

A sample of Compound 1 crystals of Form II (1 mg to 30 g) was dried for 6 hours in an oven at an isothermal 60° C. temperature under reduced pressure (1-4 Torr) using an Edwards RV8 pump (or in a vacuum oven). Sample was allowed to cool to room temperature and crystal Form III obtained was analyzed as described in Examples 3, 4, 5 and 6.

B. General Drying (Annealing) Procedures and Results:

All samples of Forms I and II were transformed to Form III without observable decomposition (by ¹H NMR, TGA, XRPD and solubility) when heated at temperatures of 60, 70, 80, 90 or 100° C. under air atmosphere. Annealing process was done above the temperature of solvent elimination. No degradation was observed up to 160° C. when crystals were annealed under nitrogen or under reduced pressure. Annealing was also shown to proceed slowly at temperatures as low as 50° C.

As an example, when temperatures of 60, 70 and 90° C. were used, subsequent DSC analysis gave melting points respectively of 185.5, 184.5 and 183.4° C., and a mass enthalpy respectively of 84.6, 66.8 and 64.9 J/g. A slight degradation (less than 4%) was observed (NMR and solubility) when a temperature of 120° C. was used (melting point of 174° C., mass enthalpy of 39.7 J/g) under air atmosphere.

When using the steps: (a) fermentation and isolation as in Example 1.2; (b) production of Form II crystals as in Example 2.2B; and (c) annealing (drying) to produce Form III as in Example 2.4A, the overall result was approximately 50 g of crystal Form III per 450 L fermentation.

Crystal Form III was also observed when Compound 1 dissolved at a concentration of about 8-10 g/L in the lower phase of a mixture of chloroform/methanol/cyclohexane/water, in a volume ratio of about 5:10:2:5 (HSCC, lower phase mobile), was concentrated to dryness on a rotavap (rotary evaporator) with gentle warming.

### Example 3

### General Characterization of Crystal Forms I, II and III

Compound 1 crystals of Forms I, II and II, prepared according to Example 2, were found to have the properties as described here and in Examples 4, 5 and 6.

In solution, no crystalline form exists, and thus the physiochemical solution characteristics, i.e. ¹H NMR spectra and ultraviolet spectra of the crystalline polymorphs and substantially pure amorphous forms of Compound 1 should be the same. The ¹H NMR spectra obtained for all crystalline forms of Compound 1 were consistent with the structure of Compound 1 and the NMR spectra described in U.S. application Ser. No. 10/762,107 filed Jan. 21, 2004, also published as WO 2004/065591 in August 2004.

In general, substantially pure crystals of Compound 1 appeared as grey to greyish-silver crystals. The appearance of the crystals depended on their purity (not their degree of crystallinity), which in general depended on the purity of Compound 1 starting material. Less pure crystal forms (e.g. 90-94%) exhibited a very light brownish color.

### Example 4

### XRPD Patterns of Crystal Forms I, II and III

4.1 General Procedure:

X-Ray powder diffraction analysis (XRPD) was performed on samples prepared according to standard procedures. X-Ray analyses were performed using a Diffractometer D5000-Siemens/Bruker AXS, using a radiation source Co 1.79091 Angstrom, and Si detection. Data were collected on a 2-4 mg sample of crystals on silicium plates, using 1-2 mg of silicium as reference standard, at room temperature without rotation of the sample, with constant shuttles at 2°/2°/0.02 mm. X-rays intensities were collected at theta angles from 3 to 700 with increment angles of 0.01° per second.

4.2 Results:

Forms I, II and III were characterized by x-ray powder diffraction patterns (XRPD) as shown, for example, in the diffractograms of FIGS. 1(a) to (d), which were collected respectively from Form III, Form II (i-PrOH/water), Form I and Form II (EtOH/Water). The values detailed in Table 1 are the most significant values and are expressed in “2-Theta Angles” in degrees (±1%) and relative intensities “RI” (S=strong, M=medium, W=weak, V=very, and combinations, for example VS=very strong).

TABLE 1
X-Ray powder diffraction (XRPD) pattern
of Crystal Forms I, II and III (±1%)

| | | |
|---|---|---|
| Form I | Form II | Form III |

| 2-θ | RI | 2-θ | RI | 2-θ | RI |
|---|---|---|---|---|---|
| 4.14 | S * | 4.16 | VS | 3.96 | VS |
| 5.14 | VS | 8.32 | M | 7.86 | W |
| 10.34 | M | 12.50 | M | 11.80 | S |
| 15.20 | S | 16.70 | M | 15.74 | M |
| 20.78 | M | 20.94 | M | 23.64 | M |
| 22.80 | W | 25.20 | S | 27.62 | M |
| 26.02 | M | 29.48 | W | — | — |
| 2-θ | RI | 2-θ | RI | 2-θ | RI |
| 31.20 | M | 33.82 | W | — | — |

* Traces of crystal Form II

Analysis of crystal (Table 1, FIG. 1(c)) obtained from methanol/water crystallization showed crystals of Form I, sometimes found to contain traces of Form II crystals.

Crystallization in either ethanol/water or isopropanol/water produced Form II crystals (Table 1, FIG. 1(d) and FIG. 1(b) respectively). Form II produced from either i-PrOH/water or EtOH/water did not show any significant differences in XRPD patterns, and are considered equivalent.

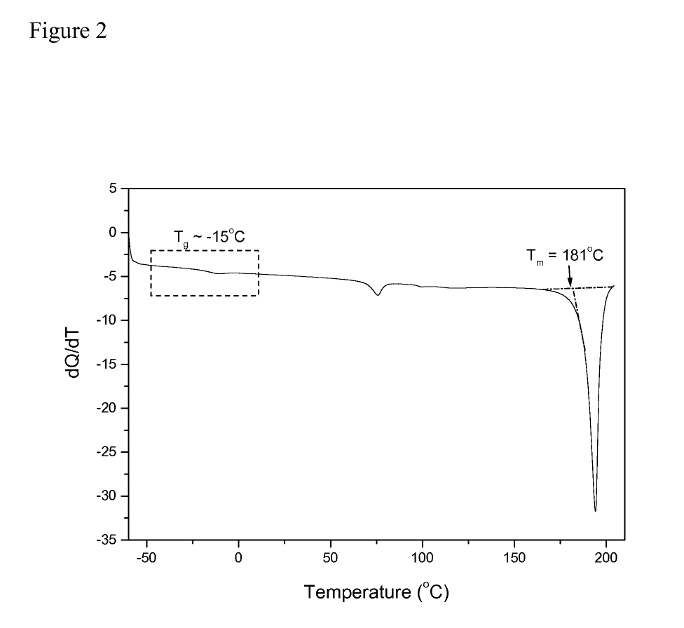

Also, all crystalline forms (Forms I and II) were found to transform into a third form (Form III) upon drying, irrespective of the solvent used for crystallization. Analysis of the XRPD results obtained for crystals post-annealing showed Form III crystals (Table 1, FIG. 1(a)) in all cases. After the annealing step, both Forms I and II were transformed into Form III crystals. Compound 1 powder, even though considered partly amorphous (see DSC experiments, Example 5 and FIG. 2), featured certain crystallinity. Its crystalline part was mostly composed of Form I crystals, and, after the first transition, part of the powder turned to Form III crystals, as observed in all other cases.

### Example 5

### DSC of Crystal Forms I, II and III

5.1 General Procedures:

Differential Scanning Calorimetry analysis were done using a TA Instruments Q1000-DSC (serial number 1000-0024) scanner with a DSC cell. A refrigerated cooling system was connected to DSC instrument, allowing the cooling of the sample down to −90° C. DSC instrument was calibrated, as recommended by the ISO Guide 25, using an Indium Metal Temperature Standard. High volume stainless steel pans (with lids and seal, TA Instrument Cat. No. 900825.902) were used as sample containers. Three different condition sets (A, B, C) were used, depending on the desired parameter or result to measure.

A. T_{g} (glass transition temperature) determination (at least partly amorphous powder) on a 10-12 mg samples were accomplished using the following conditions: cooling to −60° C. (ramp: 20° C./min); heating until 160° C. (ramp: 20° C./min); isothermal step (160° C., 30 minutes); cooling to −60° C. (ramp: 20° C./min); and heating to 210° C. (ramp: 20° C./min). The thermogram of FIG. 2 was produced using this procedure, but shows only the second heating ramp, i.e. from −60° C. to 21° C. at a temperature ramp of 20° C./min.

B. First order transitions determination (crystals and powder forms) on 5-6 mg samples was accomplished by heating from room temperature to 210° C. (ramp: 5° C./min). Melting temperature and mass enthalpy were determined on 1.5-2 mg samples using the same conditions.

C. Crystal type changes (annealing of both crystals and powder forms) were determined on 5-6 mg samples, and using the following conditions: heating until 160° C. (ramp: 5° C./min); isothermal step (160° C., 120 minutes); cooling to room temperature (ramp: 5° C./min); and heating to 210° C. (ramp: 5° C./min).

5.2 Results:

TABLE 2
Properties of Amorphous powder and Crystal Forms I, II and III

| | Powder | Form I | Form II | Form III |
|---|---|---|---|---|
| Appearance | Brownish | Silver-grey | Silver-grey | Silver-grey |
| Melting point ^{b} | 181° C. | 183° C. ^{a} | 183° C. ^{a} | 183° C. |
| Fist order | 120-145° C. | ~80° C. and | 100-140° C. | N/A |
| Transition(s) | | 100-140° C. | | |
| Glass transi- | −15° C. | N/O | N/O | N/O |
| tion (T_{g}) | | | | |

^{a} Forms I and II convert to Form III below melting point (explanation below)
^{b} onset temperature determined by DSC (±5° C.).
N/A: not applicable
N/O: not observed

Differential scanning calorimetry thermograms were done for all Forms, including the amorphous powder. Exemplary DSC scans are provided herewith (FIGS. 2 to 5), and results obtained are summarized in Table 2. DSC of amorphous powder (procedure A, FIG. 2) revealed a glass transition temperature (T_{g}) of about −15° C., which T_{g} is found to be consistent with amorphous forms of compounds bearing hydrocarbon chains, for example, a T_{g} of about −50° C. to −60° C. is expected for compounds bearing a saturated hydrocarbon chain. The value observed is lower than the standard minimum of 50° C., but more preferably 100° C., to avoid the risk of having physical state transformations overtime in oral solid pharmaceutical agents (see, for example, Bechard and Down (1992), Pharmaceutical Research, vol 9, no 4, 521-528).

In DSC thermograms of Form I (procedure B, FIG. 3(a)), generally, two first order transitions were observed below the melting point. A first transition was observed around 80° C. and a second ranging from about 100 to 140° C. The first transition observed in Form I may be caused by solvent elimination.

In DSC thermograms of Form II (procedure B, FIG. 4(a)), a first order transition was observed below the melting point. The transition was showed at a temperature ranging from about 100 to 140° C.

The transition around 100 to 140° C. observed in both Forms I and II corresponds to the 3-dimensional molecular rearrangement of the molecules to produce the more stable Form III without degradation, as shown by XRPD patterns and NMR (no degradation products).

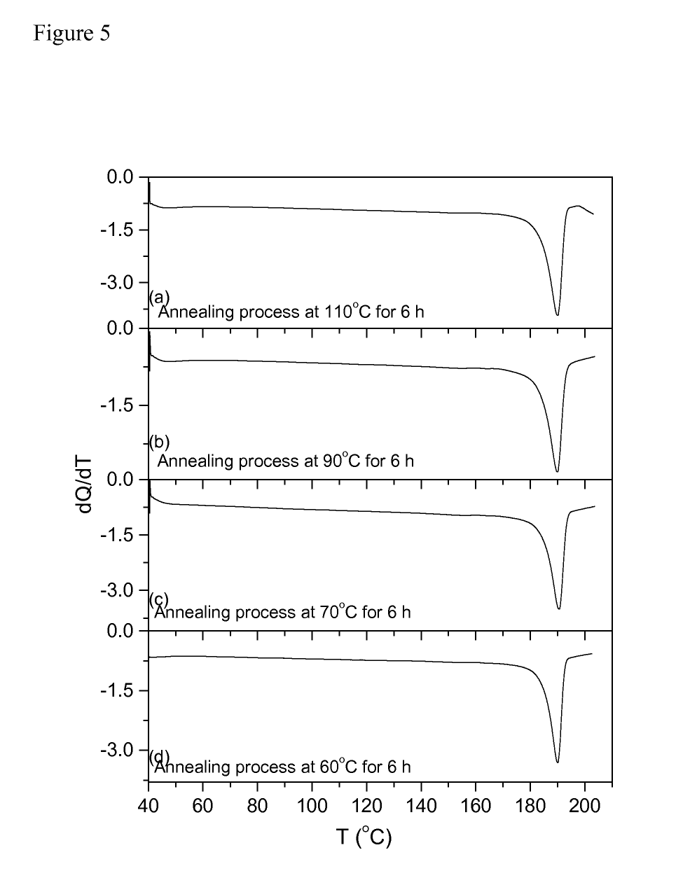

DSC thermogram of Form III (procedure B, FIGS. 3(b) and 4(b)) showed no first transition below melting point. This further confirms that the first-order transitions observed below melting point for Forms I and II were related to 3-dimensional rearrangement. DSC experiments were done on Form III crystals obtained by thermal treatment of Forms I and II at different temperatures. FIG. 5(a to d) shows the results obtained after annealing of Form II (from ethanol) respectively at 110, 90, 70 and 60° C. under reduced pressure.

Melting temperatures of Forms I, II and III all gave the same result, an onset temperature by DSC of about 183° C.±5° C. Since they convert to Form III below melting point, the melting point observed is in fact, the temperature at which Form III melts.

Other experiments such as DSC scans including an annealing step (with 160° C. isothermal, procedure C), were also done under nitrogen to further characterize crystal Forms I and II and the powder form. Observations were consistent with the results described above.

Melting temperature of Form III, when measured using a capillary U.S.P apparatus (especially designed from requirements contained in United States Pharmacopeia), gives a mean result of 184° C., with a standard deviation of 2° C.

### Example 6

### Thermogravimetric Analysis of Crystal Forms I, II and III

6.1 General Procedure:

Thermogravimetric analysis (TGA) was performed using a TA Instruments Q500-TGA (serial number: 0500-0006). The instrument was calibrated in terms of temperature and weight, as required by the Manufacturer. Weight calibration was done using a Certified Weight (Class 1 and Class E2). The temperature was calibrated using a Nickel Wire Curie Point Temperature Standard (serial number: CRM2-184). Platinum 100 μL pans (TA Instrument Catalog No. 952018.906) were used as sample containers. The data were collected using the following conditions: temperature ramp of 20° C./min from 9 to 550° C.; nitrogen flow was changed to air flow at 550° C. to facilitate oxidation and final degradation; temperature ramp 20° C./min from 550 to 700° C. in order to reach ˜100% weight loss.

6.2 Results:

Examples of results obtained from thermogravimetric analysis (TGA) of crystal Forms I, II (from ethanol/water) and III are shown in FIGS. 6 and 7. TGA of Forms I and II (e.g., FIGS. 6(b) and 7(b) respectively) showed a weight loss of about 6% before the first transition without degradation (as shown by NMR and XRPD patterns), meaning there is solvent elimination (e.g., water, methanol, ethanol or isopropanol). Weight loss occurred below 100° C. for both crystal Forms I and II.

After solvent elimination, the crystal started its transformation into another crystal type (Form III). No weight loss occurred between the end of solvent elimination and the melting point. A second weight loss for Forms I and II occurred after the melting point, which was caused by degradation of the molten compound.

TGA of Form III shown in FIGS. 6(a) and 7(a), obtained from the annealing of Form 1 or II respectively, showed no weight loss before melting point, which confirmed that solvent elimination caused the first weight loss in Forms I and II. Weight loss was observed after the melting point was reached, as for Forms I and II, showing decomposition occurred.

### Example 7

### Solubility Determination in Water

The thermodynamically most stable forms were determined by solubility testing. Generally, most stable forms exhibit lower solubility properties. The drug solubility of a sample of each of crystal Form I and Form II and two samples of Form III (obtained from drying of Form I and Form II) was evaluated in water using an HPLC-MS method (High performance liquid chromatography apparatus coupled to a mass spectrometer). Saturated aqueous solutions of the crystals were stirred and kept at ambient temperature for 24 hours. Solutions were centrifuged at 3600 rpm and an aliquot of supernatant was analyzed by HPLC-MS. The results are shown in Table 3 below.

TABLE 3
Drug solubility in Water (after 24 hours, not at equilibrium)

| Crystal Form | Solubility (μg/mL) |
|---|---|
| Form I (MeOH/water) | 6.59 |
| Form II (EtOH/water) | 3.98 |
| Form III (annealed Form I) | 0.65 |
| Form III (annealed Form II) | <LOD* |

*LOD: Limit of detection of 10 ng/mL

The results shown in Table 3 indicated that the crystal Form III was clearly more stable than crystal Forms I and II. Form III from two different sources exhibited a difference in solubility, which may be explained by the fact that solubility tests were not done at equilibrium, but for a fixed period of 24 hours.

All patents, patent applications, and published references cited herein are hereby incorporated by reference in their entirety. In case of conflict, the present specification, including definitions, will control. While this invention has been particularly shown and described with reference to preferred embodiments thereof, it will be understood by those skilled in the art that various changes in form and details may be made therein without departing from the scope of the invention encompassed by the appended claims.

1.-80. (canceled)

81. A crystalline form of Compound 1, said Compound 1 having the structural formula:

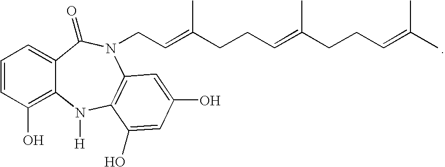

82. The crystalline form of claim 81, wherein said crystalline form produces an X-Ray diffraction pattern essentially as shown in FIG. 1(a), 1(b), 1(c) or 1(d).

83. The crystalline form of claim 81, wherein said crystalline form produces a differential scanning calorimetry (DSC) thermogram essentially as shown in FIG. 3(a), 3(b), 4(a), 4(b), 5(a), 5(b), 5(c) or 5(d).

84. The crystalline form of claim 81, wherein said crystalline form produces a thermogravimetry analysis (TGA) thermogram essentially as shown in FIG. 6(a), 6(b), 7(a), or 7(b).

85. The crystalline form of claim 81 characterized by the following angular positions (two theta angles±1%) in a X-Ray powder diffraction pattern:
a) 5.1°, 10.3°, 15.2°, 20.8°, 22.8°, 26.0° and 31.2° (Form I);
b) 4.2°, 8.3°, 12.5°, 16.7°, 20.9°, 25.2°, 29.5° and 33.8° (Form II); or
c) 4.0°, 7.9°, 11.8°, 15.7°, 23.6° and 27.6° (Form III).

86. The crystalline form of claim 81 in a substantially pure form.

87. A process for making a crystalline form of Compound 1 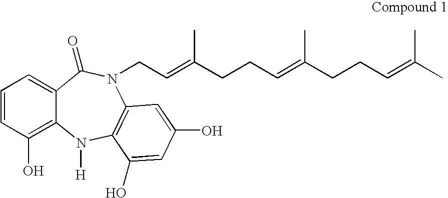
comprising:
a) treating Compound 1 with a solvent system comprising a lower alkyl alcohol under conditions inducing formation of a crystalline form of Compound 1 in the solvent system; and
b) separating the crystalline form of Compound 1 from the solvent system of a), thereby making a crystalline form of Compound 1.

88. A process for making crystalline Form III of Compound 1 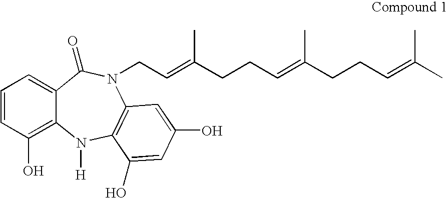
comprising:
a) treating Compound 1 with a solvent system comprising a lower alkyl alcohol under conditions inducing formation of a crystalline form of Compound 1 in the solvent system;
b) separating the crystalline form of Compound 1 from the solvent system of a), and
c) drying the crystalline form of Compound 1 obtained in b) at a temperature of about 50° C. to about 170° C., thereby making crystalline form III of Compound 1.

89. The process of claim 88, wherein said drying is done under inert conditions.

90. The process of claim 87, wherein said solvent system further comprises water and the lower alkyl alcohol is selected from the group comprising methanol, ethanol and isopropanol.

91. The process of claim 88, wherein said solvent system further comprises water and the lower alkyl alcohol is selected from the group comprising methanol, ethanol and isopropanol.

92. The process of claim 87, wherein the crystalline form of Compound 1 is Form I or Form II.

93. A pharmaceutical composition comprising a therapeutically effective amount of a crystalline form of Compound 1 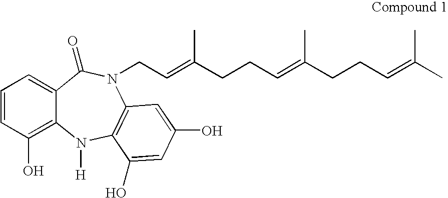
, and a pharmaceutically acceptable carrier.

94. A method of treating a neoplastic condition in a subject comprising administering to a subject having a neoplastic condition a therapeutically effective amount of a crystalline form of Compound 1

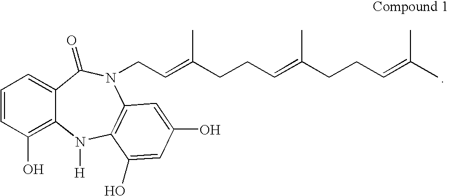

95. The method of claim 94, wherein the neoplastic condition is selected from the group consisting of lung cancer, colorectal cancer (including colon cancer), CNS cancer (including glioma), ovarian cancer, renal cancer, prostate cancer, breast cancer, hematopoietic cancer (including leukemia) and melanoma.
